(19) United States
(12) Patent Application Publication
(10) Pub. No.: US 2007/0050856 A1
(43) Pub. Date: Mar. 1, 2007

(54) Use of protein products for preventing and treating pancreatic diseases and/or obesity and/or metabolic syndrome

(76) Inventor: Daria Onichtchouk, Gottingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/489,724

(22) Filed: Jul. 20, 2006

Related U.S. Application Data
(63) Continuation-in-part of application No. PCT/EP05/00545, filed on Jan. 20, 2005.

(30) Foreign Application Priority Data
Jan. 20, 2004 (EP) 04 001 112.4

(51) Int. Cl. A01K 67/027; C12N 5/08; A61K 48/00; A61K 38/17

(52) U.S. Cl. 800/14; 435/325; 435/455; 435/368; 514/12; 514/44

(57) ABSTRACT

The present invention discloses proteins expressed by the developing pancreas, and polynucleotides, which identify and encode these proteins. The invention also relates to the use of these sequences in the diagnosis, study, prevention, and treatment of pancreatic diseases (e.g. diabetes), obesity, and/or metabolic syndrome.

This invention relates to the use of SF1-SF6 proteins, to the use of polynucleotides encoding these, and to the use of effectors/modulators thereof in the diagnosis, study, prevention, and treatment of pancreatic diseases (e.g. diabetes mellitus), obesity and/or metabolic syndrome and to the use in regeneration of tissues such as pancreatic tissues and others.

Many human proteins serve as pharmaceutically active compounds. Several classes of human proteins that serve as such active compounds include hormones, cytokines, cell growth factors, cell differentiation factors, receptors, and ligands. Most proteins that can be used as a pharmaceutically active compound fall within the family of secreted proteins. Secreted proteins are generally produced within cells at rough endoplasmic reticulum, are then exported to the golgi complex, and then move to secretory vesicles or granules, where they are secreted to the exterior of the cell via exocytosis. Examples for commercially used secreted proteins are human insulin, thrombolytic agents, interferons, interleukins, colony stimulating factors, human growth hormone, transforming growth factor beta, tissue plasminogen activator, erythropoletin, and various other proteins. Receptors of secreted proteins, which are membrane-bound proteins, also have potential as therapeutic or diagnostic agents. It is, therefore, important for developing new pharmaceutical compounds to identify secreted proteins that can be tested for activity in a variety of animal models. Thus, in light of the pervasive role of secreted proteins in human physiology, a need exists for identifying and characterizing novel functions for human secreted proteins and the genes that encode them. This knowledge will allow one to detect, to treat, and to prevent medical diseases, disorders, and/or conditions by using secreted proteins or the genes that encode them.

The pancreas is an essential organ possessing both an exocrine function involved in the delivery of enzymes into the digestive tract and an endocrine function by which various hormones are secreted into the blood stream. The exocrine function is assured by acinar and centroacinar cells that secrete various digestive enzymes via intercalated ducts into the duodenum. The functional unit of the endocrine pancreas is the islet of Langerhans. Islets are scattered throughout the exocrine portion of the pancreas and are composed of four main cell types: alpha-, beta-, delta- and PP-cells (reviewed for example in Kim S. K. and Hebrok M., (2001) Genes Dev. 15: 111-127). Beta-cells produce insulin, represent the majority of the endocrine cells and form the core of the islets, while alpha-cells secrete glucagon and are located in the periphery. Delta-cells and PP-cells are less numerous and secrete somatostatin and pancreatic polypeptide, respectively. Recently, cells producing the neuropeptide Ghrelin have been found in pancreatic islets (Wierup N. et al., (2002) Regul Pept. 107: 63-69).

Early pancreatic development has been well studied in different species, including chicken, zebrafish, and mice (for a detailed review, see Kim & Hebrok, 2001, supra). The pancreas develops from distinct dorsal and ventral anlagen. Pancreas development requires specification of the pancreas anlage along both anterior-posterior and dorsal-ventral axes. A number of transcription factors, which are critical for proper pancreatic development have been identified (see Kim & Hebrok, 2001, supra; Wilson M. E. et al., (2003) Mech Dev. 120: 65-80).

In humans, the acinar and ductal cells retain a significant proliferative capacity that can ensure cell renewal and growth, whereas the islet cells become mostly mitotically inactive. This is in contrast to rodents where beta-cell replication is an important mechanism in the generation of new beta cells. It has been suggested, that during embryonic development, pancreatic islets of Langerhans originate from differentiating duct cells or other cells with epithelial morphology (Bonner-Weir S. and Sharma A, (2002) J Pathol. 197: 519-526; Gu G. et al., (2003) Mech Dev. 120: 35-43). In adult humans, new beta cells arise in the vicinity of ducts (Butler A. E. et al., (2003) Diabetes 52: 102-110; Bouwens L. and Pipeleers D. G., (1998) Diabetologia 41: 629-633). However, also an intra-islet location or an origin in the bone marrow has been suggested for precursor cells of adult beta cells (Zulewski H. et al., (2001) Diabetes 50: 521-533; Ianus A et al., (2003) J Clin Invest. 111: 843-850). Pancreatic islet growth is dynamic and responds to changes in insulin demand, such as during pregnancy or during the increase in body mass occurring during childhood. In adults, there is a good correlation between body mass and islet mass (Yoon K. H. et al., (2003) J Clin Endocrinol Metab. 88: 2300-2308).

Pancreatic beta-cells secrete insulin in response to rising glucose levels and other secretagogues such as arginine. Insulin amongst other hormones plays a key role in the regulation of the fuel metabolism. Insulin leads to the storage of glycogen and triglycerides and to the synthesis of proteins. The entry of glucose into muscles and adipose cells is stimulated by insulin. In patients who suffer from diabetes mellitus type 1 or LADA (latent autoimmune diabetes in adults) (Pozzilli P. and Di Mario U., (2001) Diabetes Care. 8: 1460-1467) beta-cells are being destroyed due to autoimmune attack. The amount of insulin produced by the remaining pancreatic islet cells is too low, resulting in elevated blood glucose levels (hyperglycemia). In diabetes type 2, liver and muscle calls loose their ability to respond to normal blood insulin levels (insulin resistance). High blood glucose levels (and also high blood lipid levels) lead to an impairment of beta-cell function and to an increase in beta-cell apoptosis. It is interesting to note that the rate of beta-cell neogenesis does not appear to change in type 2 diabetics (Butler et al., 2003 supra), thus causing a reduction in total beta-cell mass over time. Eventually the application of exogenous insulin becomes necessary in type 2 diabetics.

Improving metabolic parameters such as blood sugar and blood lipid levels (e.g. through dietary changes, exercise, medication or combinations thereof) before beta cell mass has fallen below a critical threshold leads to a relatively rapid restoration of beta cell function. However, even after such a treatment the pancreatic endocrine function would remain impaired due to the only slightly increased regeneration rate. Treatments which increase the rate of neogenesis will have a beneficial effect due to enhanced insulin secretory capacity.

In type 1 diabetics, where beta-cells are being destroyed by autoimmune attack treatments have been devised which modulate the immune system and may be able to stop or strongly reduce islet destruction (Raz I. et al., (2001) Lancet 358: 1749-1753; Chatenoud L. et al., (2003) Nat Rev Immunol. 3: 123-132, Homann D. et al., (2002) Immunity 3: 403-415). However, due to the relatively slow regeneration of human beta cells such treatments could only be fully successful at improving the diabetic condition if they are combined with an agent which can stimulate beta cell regeneration.

Thus, both for type 1 and type 2 diabetes (early and late stages) there is a need to find novel agents which stimulate beta cell regeneration.

A variety of model organisms has been used to study the formation of beta cells and to analyze the effect of treatments aimed at the improvement of diabetic conditions. Zebrafish has become a popular model vertebrate for the study of developmental processes as well as for pharmacological and toxicological studies over the last decade (Rubinstein A. L., (2003) Curr Opin Drug Discov Devel. 6: 218-223; Grunwald D. J. and Eisen J. S., (2002) Nat Rev Genet. 3: 717-724). In this organism, large numbers of transparent embryos which rapidly develop outside of their mother are readily available. Transgenic lines expressing fluorescent proteins under the control of tissue-specific promoters allow to rapidly assess the effects of pharmacological treatments or gene loss- and gain-of-function treatments. Zebrafish islets contain the same cell types in a similar spatial organization as mammalian islets. A large number of genes which control pancreatic development in mammals also control pancreatic development in zebrafish (Biemar F. et al., (2001) Dev Biol. 230: 189-203; Ober E. A. et al., (2003) Mech Dev. 120: 5-18). Suppressing gene function in zebrafish embryos using antisense oligonucleotides, modified Peptide Nucleic Acids (mPNAs) or other antisense compounds with good efficiency and specificity yields phenotypes which are usually indistinguishable from genetic mutants in the same gene (Nasevicius A. and Ekker S. C., (2000) Nat Genet. 26: 216-220; Effimov et al., NAR 26; 566-575; Urtishak K. A: et al., 5th international conference on zebrafish development and genetics, Madison/Wis. 2002, abstr. #17). Thus, zebrafish embryos represent a relevant model to identify genes or compounds which control beta cell formation in humans.

Diabetes is a very disabling disease, because today's common anti-diabetic drugs do not control blood sugar levels well enough to completely prevent the occurrence of high and low blood sugar levels. Out of range blood sugar levels are toxic and cause long-term complications like for example renopathy, retinopathy, neuropathy, and peripheral vascular disease. There are also a host of related conditions, such as obesity, hypertension, heart disease, and hyperlipidemia, for which persons with diabetes are at substantially increased risk.

Apart from the impaired quality of life for the patients, the treatment of diabetes and its long-term complications presents an enormous financial burden to our healthcare systems with rising tendency. Thus, for the prevention or treatment of, type 1 and type 2 diabetes as well as for latent autoimmune diabetes in adults (LADA) there is a strong need in the art to identify factors that induce regeneration of pancreatic insulin producing beta-cells. These factors could restore normal function of the endocrine pancreas once its function is impaired or event could prevent the development or progression of diabetes type 1, diabetes type 2, or LADA.

Obesity is one of the most prevalent metabolic disorders in the world. It is still a poorly understood human disease that becomes as a major health problem more and more relevant for western society. Obesity is defined as a body weight more than 20% in excess of the ideal body weight frequently resulting in a significant impairment of health. Obesity may be measured by body mass index, an indicator of adiposity or fatness. Further parameters for defining obesity are waist circumferences, skinfold thickness and bioimpedance. It is associated with an increased risk for cardiovascular disease, hypertension, diabetes mellitus type 2, hyperlipidaemia and an increased mortality rate. Obesity is influenced by genetic, metabolic, biochemical, psychological, and behavioral factors and can be caused by different reasons such as non-Insulin dependent diabetes, increase in triglycerides, increase in carbohydrate bound energy and low energy expenditure (Kopelman P. G., (2000) Nature 404: 635-643).

The concept of ‘metabolic syndrome’ (syndrome x, insulin-resistance syndrome, deadly quartet) was first described 1966 by Camus and reintroduced 1988 by Reaven (Camus J. P., (1966) Rev Rhum Mal Osteoartic 33: 10-14; Reaven G. M., (1988), Diabetes 37: 1595-1607). Today, metabolic syndrome is commonly defined as clustering of cardiovascular risk factors like hypertension, abdominal obesity, high blood levels of triglycerides and fasting glucose as well as low blood levels of HDL cholesterol. Insulin resistance greatly increases the risk of developing the metabolic syndrome (Reaven G., (2002) Circulation 106: 286-288). The metabolic syndrome often precedes the development of type 2 diabetes and cardiovascular disease (Lakka H. M. et al., (2002) JAMA 288: 2709-2716). The control of blood lipid levels and blood glucose levels is essential for the treatment of the metabolic syndrome (see, for example, Santomauro A. T. et al., (1999) Diabetes, 48: 1836-1841).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms and/or molecules influencing obesity or body weight/body mass regulations are not known.

There is a need in the prior art for the identification of candidate genes that are specifically expressed in early development in certain pancreatic tissues. These genes and the thereby encoded proteins can provide tools to the diagnosis and treatment of severe pancreatic disorders and related diseases. Therefore, this invention describes proteins that are specifically expressed in pancreatic tissues early in the development. The invention relates to the use of these genes and proteins in the diagnosis, prevention and/or treatment of pancreatic dysfunctions, such as diabetes, and other related diseases such as obesity and/or metabolic syndrome. These proteins and genes are especially useful in regeneration processes, such as regeneration of the pancreas cells.

In this invention, we disclose proteins referred to as SF1, SF2, SF3, SF4, SF5 and SF6 (SF1-SF6), which are involved in pancreas development, regeneration, and in the regulation of energy homeostasis. SF1-SF6 corresponds to mammalian proteins as described in Table 2.

SF1 was described as a cysteine-type endopeptidase and might be a glucocorticosteroid-regulated lipocalin. Glucocorticosterolds are known to play a negative role in insulin secretion in pancreatic cells.

The SF2 protein was described to contain seven transmembrane domains, but to be structurally and functionally distinct from G-protein-coupled receptors. SF2 was shown to be expressed in skeletal muscle and in pancreas. The SF2 protein contains an extracellular C-terminal part.

SF3 is a 32-kDa glycoprotein, which participates in the regulation of morphogenesis and cellular differentiation through its modulation of cell-matrix interactions. SF3 is an extracellular calcium binding glycoprotein that is involved in maintaining the cell matrix. SF3 associates with various integral proteins of the extracellular matrix, including thrombospondin 1, vitronectin and fibrillar collagens, during normal cell migration, morphogenesis and differentiation. SF3 modulates the expression of extracellular metalloproteinases, and it is required for granulation and tissue formation during normal repair of skin wounds. In the adult, SF3 is expressed primarily in regions, which rapidly divide and turnover, and at sites of injury and disease.

SF4 is a growth factor that belongs to the Tgf beta superfamily. SF4 is the closest homolog of the Xenopus genes derriere and Vg1 involved in endoderm specification.

SF5 is a serine carboxypeptidase that is expressed in aorta, bladder, and kidney.

SF6 belongs to the ADP-ribosylhydrolase family. The function of this family of proteins is reverse ADP ribosylation, removing ADP ribose moiety from a specific amino acid of target protein. An ADP-ribosylation cycle nay play a regulatory role in vertebrate tissues, e.g. regulating cell-matrix or cell-cell interactions. In lymphocytes, ADP-ribosylation of surface proteins is associated with changes in p56lck tyrosine kinase-mediated signaling. The physiological role for the SF6 protein is unknown. SF6 is expressed in pancreas, T and B cell lines, neiroblastoma, fetal brain, fetal liver.

Accordingly, the present invention relates to proteins with novel functions in the human metabolism, regeneration, and pancreatic developmental processes.

The present invention discloses specific genes and proteins encoded thereby and effectors/modulators thereof involved in the regulation of pancreatic function and metabolism, especially in pancreas diseases such as diabetes mellitus, e.g. insulin dependent diabetes mellitus and/or non insulin dependent diabetes mellitus, and/or LADA and/or metabolic syndrome, obesity, and/or related disorders such as coronary heart disease, eating disorder, cachexia, hypertension, hypercholesterolemia (dyslipidemia), liver fibrosis, and/or gallstones. Further, the present invention discloses specific genes and proteins encoded thereby and effectors/modulators thereof involved in the regeneration of pancreatic cells or tissues, e.g. cells having exocrinous functions such as acinar cells; centroacinar cells and/or ductal cells and/or cells having endocrinous functions, particularly cells in Langerhans islets such as alpha-, beta-, delta- and/or PP-cells, more particularly beta-cells.

In this invention, we used a screen for factors expressed in developing mammalian (mouse) pancreas, as described in more detail in the Examples. section (see Example 1). This screen identified SF1-SF6 as factors expressed in developing mouse pancreas. The present invention describes mammalian SF1-SF6 proteins and the polynucleotides encoding these, in particular human SF1-SF6, as being involved in the conditions and processes mentioned above.

The present invention relates to SF1-SF6 polynucleotides encoding polypeptides with novel functions in the development and regeneration of pancreatic tissues and thus in mammalian pancreatic diseases (e.g. diabetes), and also in body-weight regulation, energy homeostasis, and obesity, fragments of said polynucleotides, polypeptides encoded by said polynucleotides or fragments thereof. The invention also relates to vectors, host cells, and recombinant methods for producing the polypeptides and polynucleotides of the invention. The invention also relates to effectors/modulators of SF1-SF6 polynucleotides and/or polypeptides, e.g. antibodies, biologically active nucleic acids, such as antisense molecules, RNAi molecules or ribozymes, aptamers, peptides or low-molecular weight organic compounds recognizing said polynucleotides or polypeptides.

SF1-SF6 homologous proteins and nucleic acid molecules coding therefore are obtainable from vertebrate species. Particularly preferred are nucleic acids encoding the human SF1-SF6 protein and variants thereof. The invention particularly relates to a nucleic add molecule encoding a polypeptide contributing to regulating the energy homeostasis and the mammalian metabolism, wherein said nucleic acid molecule comprises
(a) the nucleotide sequence of human SF1-SF6 and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of the human SF1-SF6 proteins,
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of 15-25 bases, preferably 25-35 bases, more preferably 35-50 bases and most preferably at least 50 bases.

The function of the proteins of the invention in mammalian metabolism was validated by analyzing the expression of the transcripts in different tissues and by analyzing the role in adipocyte differentiation (see Examples for more detail).

Expression profiling studies confirm the particular relevance of the proteins of the invention as regulators of energy metabolism in mammals. We used mouse models of insulin resistance and/or diabetes, such as mice carrying gene knockouts in the leptin pathway (for example, ob/ob (leptin) or db (leptin receptor/ligand) mice) to study the expression of the proteins of the present invention. Such mice develop typical symptoms of diabetes, show hepatic lipid accumulation and frequently have increased plasma lipid levels (see Bruning J. C. et al., (1998) Mol. Cell. 2: 559-569). Expression of the mRNA of the invention was also examined in susceptible wild type mice (for example, C57BI/6) that show symptoms of diabetes, lipid accumulation, and high plasma lipid levels, if fed a high fat diet.

In addition to the expression studies in mammalian cells, functional assays were performed with SF1, SF2, SF3, SF4, SF5, and SF6. For example, in one embodiment of the invention, a functional assay was done with zebrafish. Zebrafish was selected as model because the pancreatic development in zebrafish is controlled by the same pathways as in mammals (for example, Pdx1 and Nkx2.2 at 14 hours, Islet-1, Pax4, Insulin at 16 hours). Another advantage is the transparency of the zebrafish embryos. Transgenic reporter strains allow fast analysis. Antisense oligonucleotides were designed for SF1, SF2, SF3, SF4, SF5, and SF6 (see Example 3) to study the function of the SF1, SF2, SF3, SF4, SF5, and SF6 genes in zebrafish.

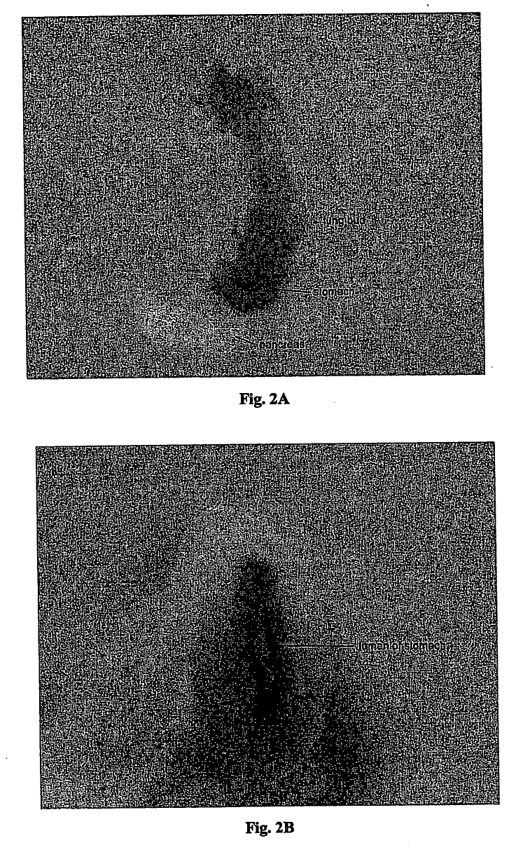

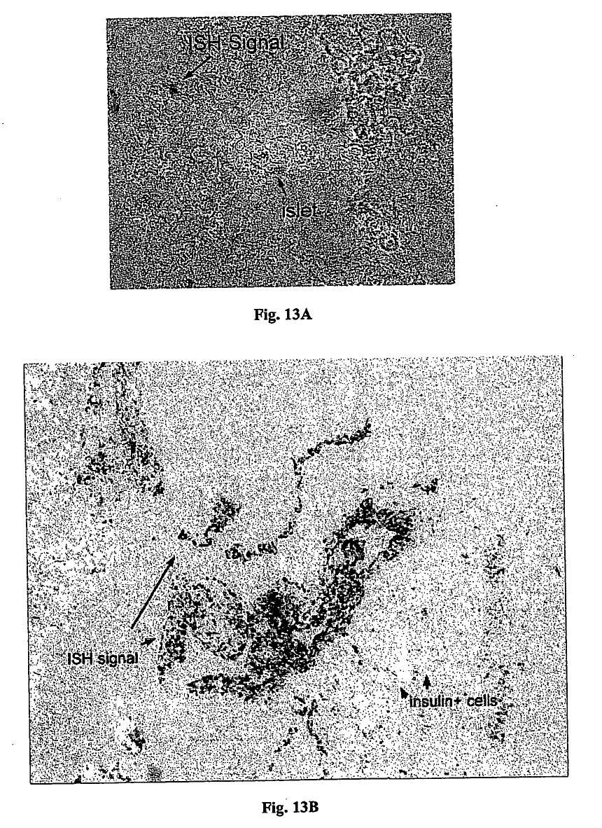

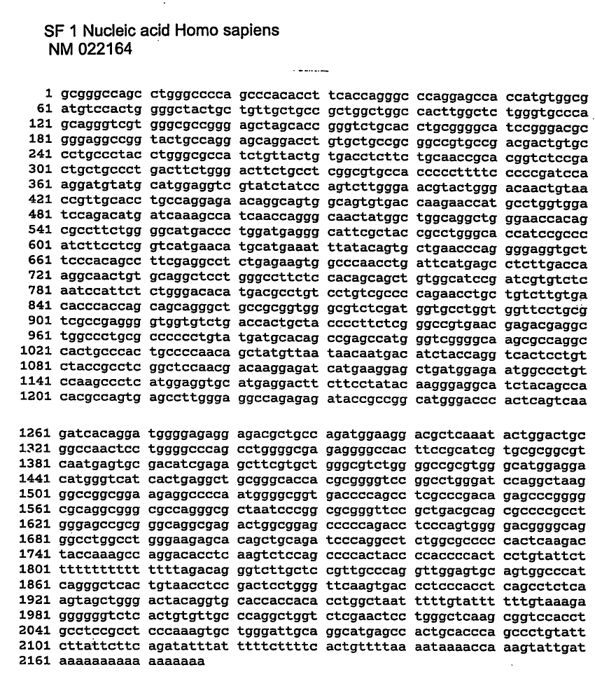

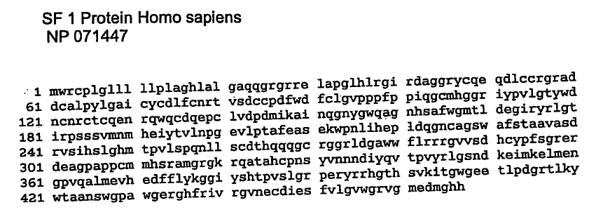

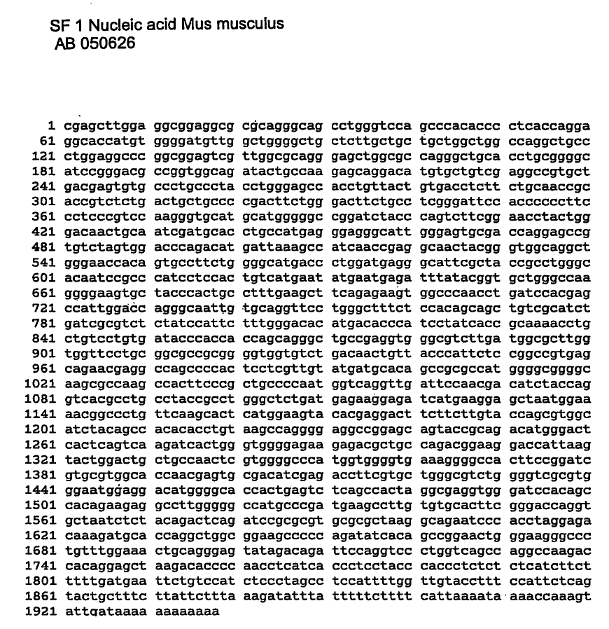

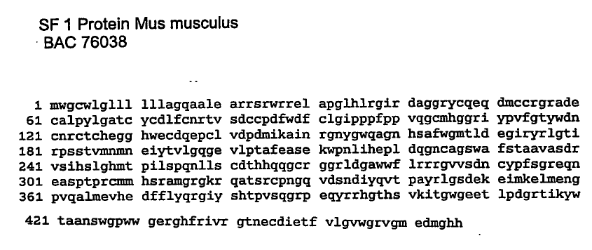

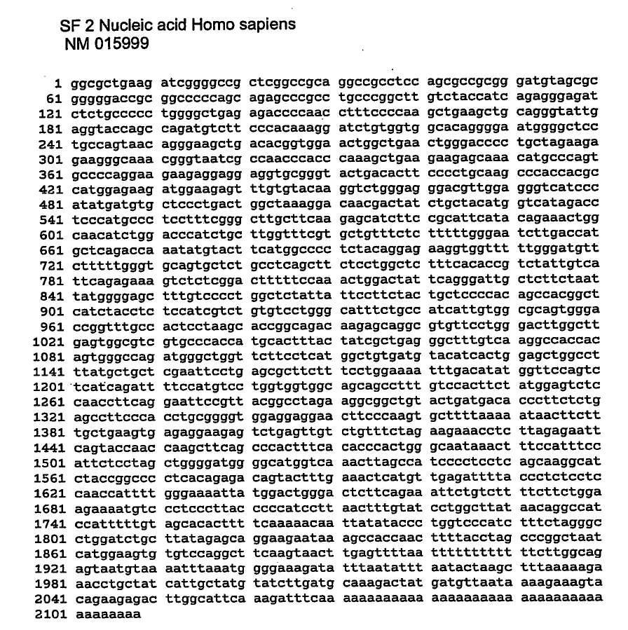

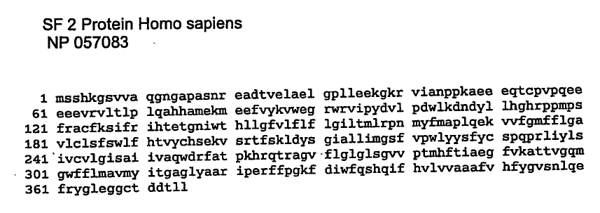

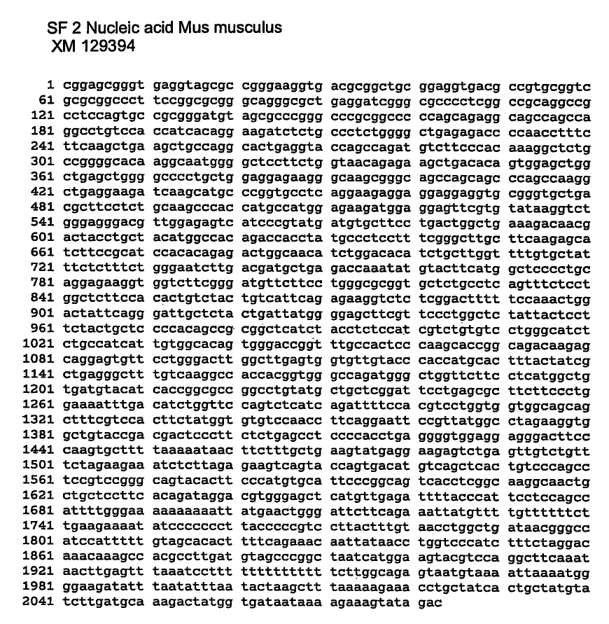

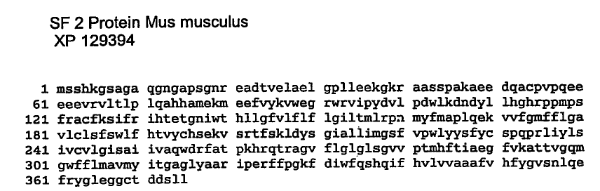

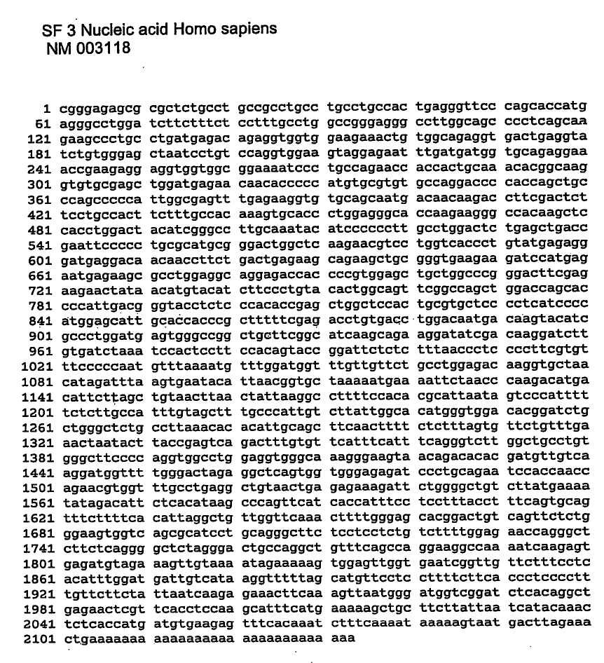

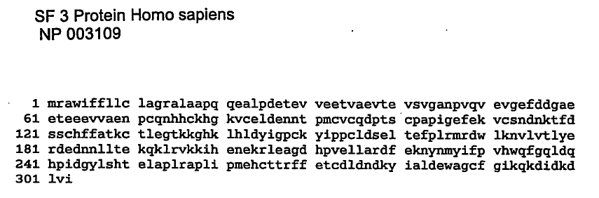

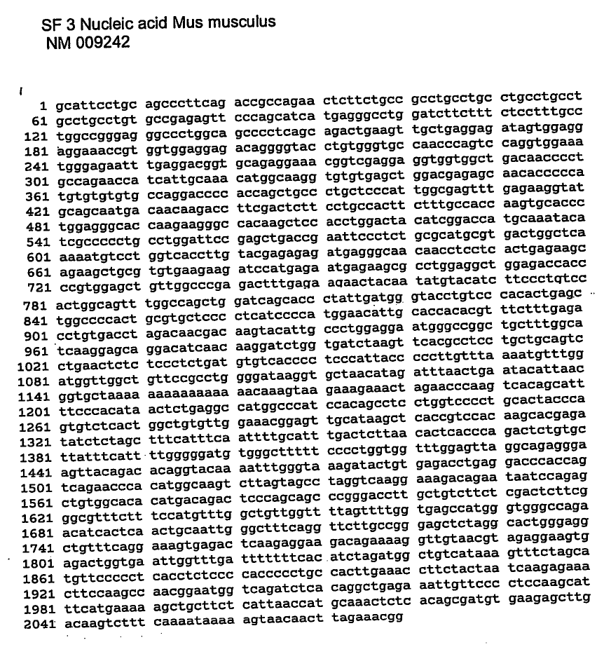

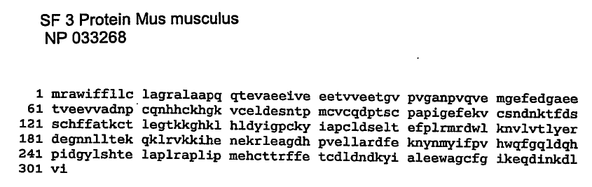

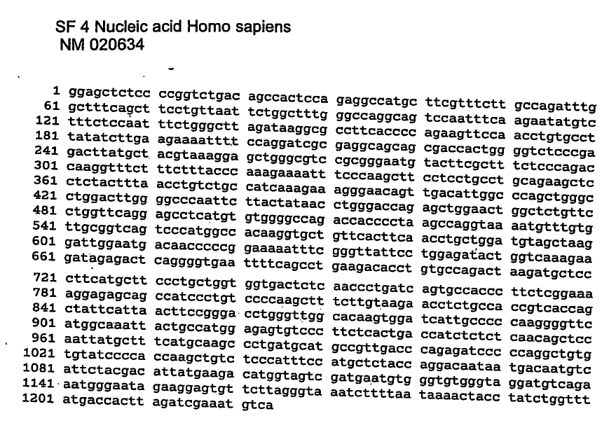

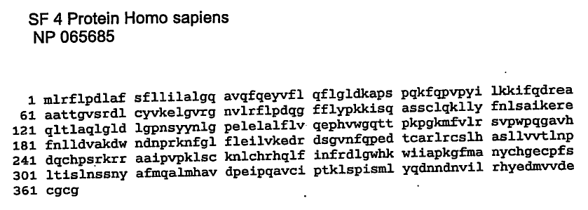

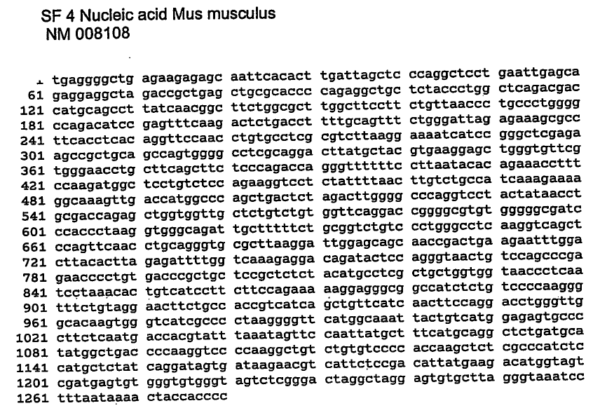

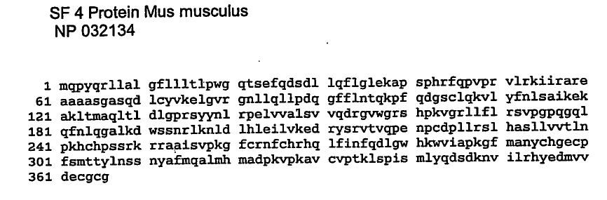

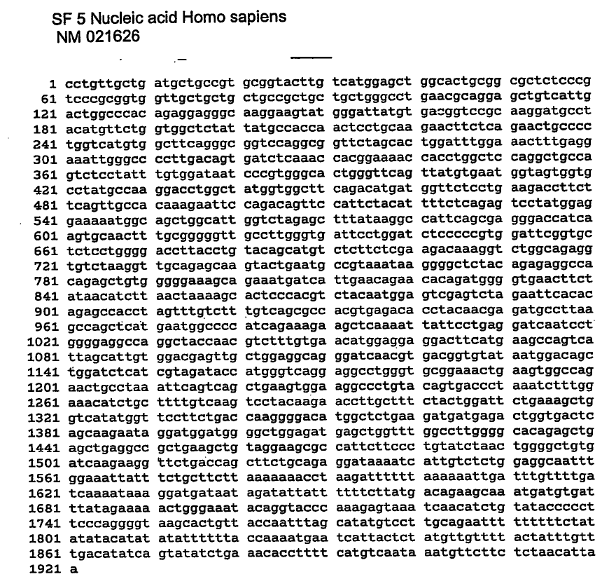

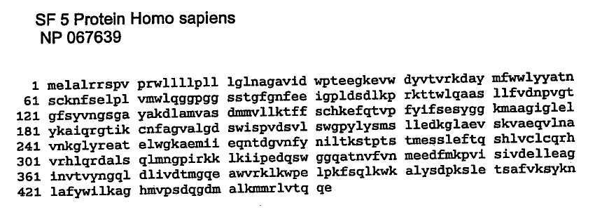

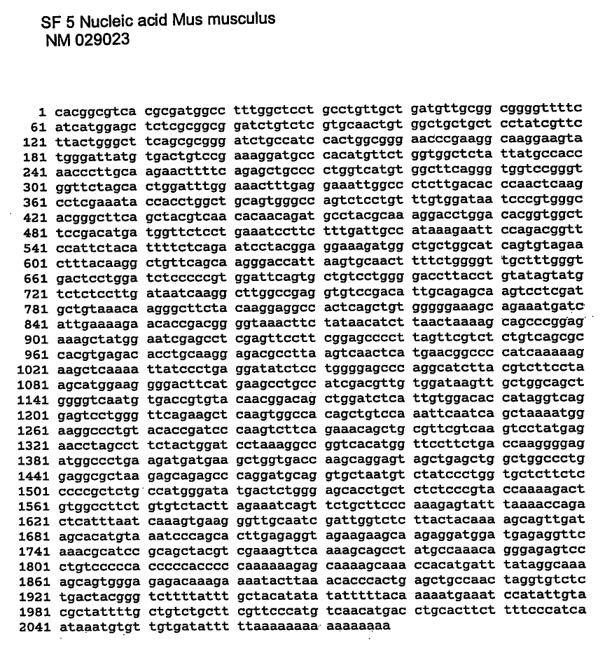

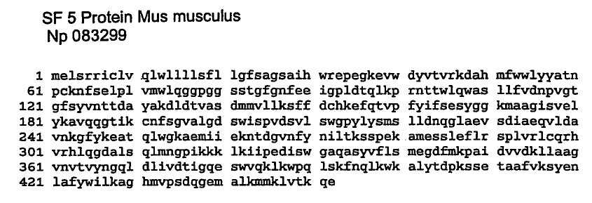

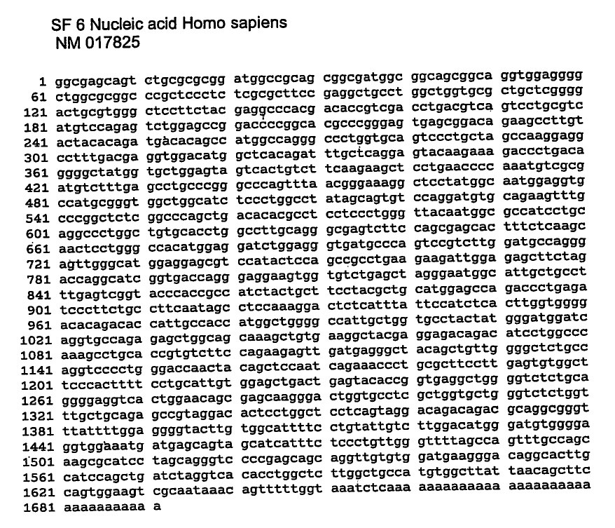

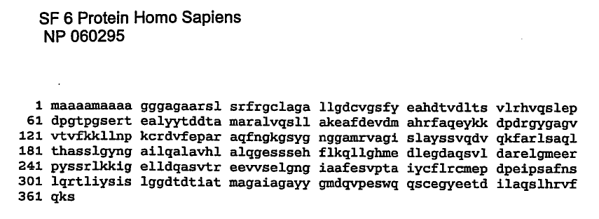

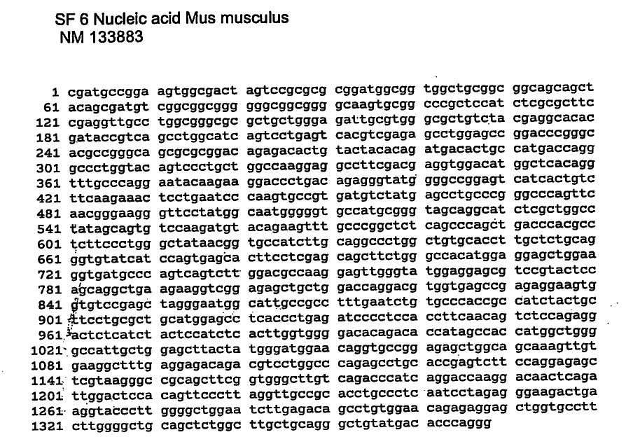

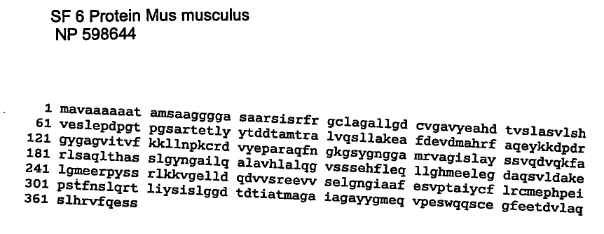

Whole-mount in situ hybridizations were performed according to standard protocols as known to those skilled in the art and as described previously (for example, Pelton, R. W. et al., (1990) Development 110,609-620; Belo, J. A. et al., (1997) Mech. Dev. 68, 45-57). The nucleic acid sequence encoding the mouse SF1 protein is expressed in the stomach and the inner organs, with a weak expression in pancreas (see FIG. 2). The nucleic acid sequence encoding mouse SF3 protein is expressed in the pancreatic tissue (see FIG. 13).

The invention also encompasses a novel use of polynucleotides that encode the proteins of the invention and homologous proteins. Accordingly, any nucleic acid sequence, which encodes the amino acid sequences of the proteins of the invention and homologous proteins, can be used to generate recombinant molecules that express the proteins of the invention and homologous proteins. In a particular embodiment, the invention encompasses a nucleic acid encoding SF1-SF6. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. The invention contemplates each and every possible variation of nucleotide sequence that can be made by selecting combinations based on possible codon choices.

Also encompassed by the invention is the use of polynucleotide sequences that are capable of hybridizing to the claimed nucleotide sequences, and in particular, those of the polynucleotide encoding the proteins of the invention, under various conditions of stringency. Hybridization conditions are based on the melting temperature (Tm) of the nucleic acid binding complex or probe, as described in Wahl G. M. et al., (1987; Methods Enzymol. 152: 399-407) and Kimmel A. R. (1987; Methods Enzymol. 152: 507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 65° C., particularly for 1 h in 0.2×SSC and 0.1% SDS at 50° C., preferably at 55° C., more preferably at 62° C. and most preferably at 65° C., a positive hybridization signal is observed. Altered nucleic acid sequences encoding the proteins which are encompassed by the invention include deletions, insertions or substitutions of different nucleotides resulting in a polynucleotide that encodes the same or a functionally equivalent protein.

The encoded proteins may also contain deletions, insertions or substitutions of amino acid residues, which produce a silent change and result in functionally equivalent proteins. Deliberate amino acid substitutions may be made on the basis of similarity in polarity, charge, solubility, hydrophobicity, hydrophilicity, and/or the amphipathic nature of the residues as long as the biological activity of the protein is retained. Furthermore, the invention relates to peptide fragments of the proteins or derivatives thereof such as cyclic peptides, retro-inverso peptides or peptide mimetics having a length of at least 4, preferably at least 6 and up to 50 amino acids.

Also included within the scope of the present invention are alleles of the genes encoding the proteins of the invention and homologous proteins. As used herein, an ‘allele’ or ‘allelic sequence’ is an alternative form of the gene, which may result from at least one mutation in the nucleic acid sequence. Alleles may result in altered mRNAs or polypeptides whose structures or function may or may not be altered. Any given gene may have none, one or many allelic forms. Common mutational changes, which give rise to alleles, are generally ascribed to natural deletions, additions or substitutions of nucleotides. Each of these types of changes may occur alone or in combination with the others, one or more times in a given sequence.

The nucleic acid sequences encoding SF1-SF6 and homologous proteins may be extended utilizing a partial nucleotide sequence and employing various methods known in the art to detect upstream sequences such as promoters and regulatory elements.

In order to express a biologically active protein, the nucleotide sequences encoding the proteins or functional equivalents, may be inserted into appropriate expression vectors, i.e., a vector which contains the necessary elements for the transcription and translation of the inserted coding sequence. Methods, which are well known to those skilled in the art, may be used to construct expression vectors containing sequences encoding the proteins and the appropriate transcriptional and translational control elements. Regulatory elements include for example a promoter, an initiation codon, a stop codon, a mRNA stability regulatory element, and a polyadenylation signal. Expression of a polynucleotide can be assured by (i) constitutive promoters such as the Cytomegalovirus (CMV) promoter/enhancer region, (ii) tissue specific promoters such as the insulin promoter (see, Soria B. et al., (2000), Diabetes 49: 157-162), SOX2 gene promoter (see Li M. et al., (1998) Curr. Biol. 8: 971-974), Msi-1 promoter (see Sakakibara S. and Okano H., (1997) J. Neuroscience 17: 8300-8312), alpha-cardia myosin heavy chain promoter or human atrial natriuretic factor promoter (Klug M. G. et al., (1996) J. Clin. Invest 98: 216-224; Wu J. et al., (1989) J. Biol. Chem. 264: 6472-6479) or (iii) inducible promoters such as the tetracycline inducible system. Expression vectors can also contain a selection agent or marker gene that confers antibiotic resistance such as the neomycin, hygromycin or puromycin resistance genes. These methods include in vitro recombinant DNA techniques, synthetic techniques, and in vivo genetic recombination. Such techniques are described in Sambrook J. et al., (1989) Molecular Cloning, A Laboratory Manual, Cold Spring Harbor Press, Plainview, N.Y. and Ausubel F. M. et al., (1989) Current Protocols in Molecular Biology, John Wiley & Sons, New York, N.Y.

In a further embodiment of the invention, natural, modified or recombinant nucleic acid sequences encoding the proteins of the invention and homologous proteins may be ligated to a heterologous sequence to encode a fusion protein. Heterologous sequences are preferably located at the N- and/or C-terminus of the fusion protein.

A variety of expression vector/host systems, as known in the art, may be utilized to contain and express sequences encoding the proteins or fusion proteins. These include, but are not limited to, micro-organisms such as bacteria transformed with recombinant bacteriophage, plasmid or cosmid DNA expression vectors; yeast transformed with yeast expression vectors; insect cell systems infected with virus expression vectors (e.g., baculovirus, adenovirus, adeno-associated virus, lentivirus, retrovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences of the invention in a sample can be detected by DNA-DNA or DNA-RNA hybridization and/or amplification using probes or portions or fragments of said polynucleotides. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of RNA probes, PCR amplification using a labeled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

The presence of SF1-SF6 in a sample can be determined by immunological methods or activity measurement. A variety of protocols for detecting and measuring the expression of proteins, using either polyclonal or monoclonal antibodies specific for the protein or reagents for determining protein activity are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton R. et al. (1990; Serological Methods, a Laboratory Manual, APS Press, St Paul, Minn.) and Maddox D. E. et al. (1983; J. Exp. Med. 158: 1211-1226).

Suitable reporter molecules or labels, which may be used, include radionuclides, enzymes, fluorescent, chemiluminescent or chromogenic agents as well as substrates, co-factors, inhibitors, magnetic particles, and the like.

The nucleic acids encoding the proteins of the invention can be used to generate transgenic animal or site specific gene modifications in cell lines. Transgenic animals may be made through homologous recombination, where the normal locus of the genes encoding the proteins of the invention is altered. Alternatively, a nucleic acid construct is randomly integrated into the genome. Vectors for stable integration include plasmids, retroviruses and other animal viruses, YACs, and the like. The modified cells or animal are useful in the study of the function and regulation of the proteins of the invention. For example, a series of small deletions and/or substitutions may be made in the genes that encode the proteins of the invention to determine the role of particular domains of the protein, functions in pancreatic differentiation, etc.

Specific constructs of interest include anti-sense molecules, which will block the expression of the proteins of the invention, or expression of dominant negative mutations. A detectable marker, such as for example lac-Z, may be introduced in the locus of the genes of the invention, where up-regulation of expression of the genes of the invention will result in an easily detected change in phenotype.

One may also provide for expression of the genes of the invention or variants thereof in cells or tissues where it is not normally expressed or at abnormal times of development in addition, by providing expression of the proteins of the invention in cells in which they are not normally produced, one can induce changes in cell behavior.

DNA constructs for homologous recombination will comprise at least portions of the genes of the invention with the desired genetic modification, and will include regions of homology to the target locus. DNA constructs for random integration need not include regions of homology to mediate recombination. Conveniently, markers for positive and/or negative selection are included. Methods for generating cells having targeted gene modifications through homologous recombination are known in the art. For non-human embryonic stem (ES) cells, an ES cell line may be employed, or embryonic cells may be obtained freshly from a host, e.g. mouse, rat, guinea pig etc. Such cells are grown on an appropriate fibroblast-feeder layer or grown in presence of leukemia inhibiting factor (LIF).

The data disclosed in this invention show that the SF1-SF6 nucleic acids and proteins and effector/modulator molecules thereof are useful in diagnostic and therapeutic applications implicated, for example, but not limited to, pancreatic diseases (e.g. diabetes mellitus, such as insulin dependent diabetes mellitus and/or non insulin dependent diabetes mellitus and/or LADA), obesity, metabolic syndrome, eating disorder, cachexia, hypertension, coronary heart disease, hypercholesterolemia (dyslipidemia), and/or gallstones. Further, the data show that the SF1-SF6 nucleic acids and proteins and effector/modulator molecules thereof are useful for the modulation, e.g. stimulation of pancreatic development, and/or for the regeneration of pancreatic cells or tissues, e.g. cells having exocrinous functions such as acinar cells, centroacinar cells and/or ductal cells and/or cells having endocrinous functions, particularly cells in Langerhans islets such as alpha-, beta-, delta- and/or PP-cells, more particularly beta-cells. Hence, diagnostic and therapeutic uses for the proteins of the invention nucleic acids and proteins of the invention are, for example but not limited to, the following: (i) tissue regeneration in vitro and in vivo (regeneration for all these tissues and cell types composing these tissues and cell types derived from these tissues), (ii) small molecule drug target, (iii) antibody target (therapeutic, diagnostic, drug targeting/cytotoxic antibody), (iv) diagnostic and/or prognostic marker, (v) protein therapy, (vi) gene therapy (gene delivery/gene ablation), and/or (vii) research tools.

For example, but not limited to, cDNAs encoding the proteins of the invention and particularly their human homologues may be useful in stimulating, enhancing or regulating the regeneration of tissues, and the proteins of the invention and particularly their human homologues may be useful when administered to a subject in need thereof. By way of non-limiting example, the compositions of the present invention will have efficacy for treatment of patients suffering from, for example, pancreatic diseases (e.g. diabetes), obesity, and for metabolic syndrome as described above.

In one embodiment of the invention, administration of SF1-SF6 nucleic acids and proteins and/or effectors/modulators thereof in a pharmaceutical composition to a subject in need thereof, particularly a human patient lead to an at least partial regeneration of, for example, pancreas cells. The composition will then at least partially restore normal pancreatic function. In one example, these cells are beta cells of the islets which will contribute to the improvement of a diabetic state. With the administration of this composition on a short term or regular basis, an increase in beta cell mass can be achieved. This effect upon the body reverses the condition of diabetes partially or completely. As the subject's blood sugar level improves, the dosage administered may be reduced in strength. In at least some cases further administration can be discontinued entirely and the subject continues to produce a normal amount of insulin without further treatment. The subject is thereby not only treated but cured entirely of a diabetic condition. However, even moderate improvements in beta cell mass can lead to a reduced requirement for exogenous insulin, improved glycemic control and a subsequent reduction in diabetic complications. In another example, other cells of the pancreas can be regenerated in vivo or in vitro to cure a certain condition. Beside diabetes, the compositions of the present invention will also have efficacy for treatment of patients with other pancreatic diseases such as pancreatic cancer, dysplasia, or pancreatitis.

The SF1-SF6 nucleic acids and proteins and effectors/modulators thereof are useful in diagnostic and therapeutic applications implicated in various embodiments as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, pancreatic diseases (e.g. diabetes), obesity, and/or metabolic syndrome as described above.

The SF1-SF6 nucleic acids and proteins and effectors/modulators thereof may be administered either as a monotherapy or as a combination therapy with other pharmaceutical agents. For example, they may be administered together with other pharmaceutical agents suitable for the treatment or prevention of pancreatic diseases and/or obesity and/or metabolic syndrome. Further, they may be administered together with pharmaceutical agents which have an immunosuppressive activity, e.g. antibodies, polypeptides and/or peptidic or non-peptidic low molecular weight substances. Preferred examples of immunosuppressive agents are listed in the following Table 1.

TABLE 1
Exemplary agents for immune suppression

| Names | Mechanism |
|---|---|
| 2-amino-1,3-propanediol | Used for preventing or treating |
| derivatives | chronic rejection in a patient |
| | receiving an organ or tissue |
| | allo- or xenotransplant |
| 2-amino-2[2-(4- | Immunosuppression, from acceler- |
| octylphenyl)ethyl] | ated lymphocyte homing |
| propane-1,3-diol | |
| hydrochloride | |
| 40-O-(2-hydroxyethyl)- | Sirolimus (rapamycin) derivative, |
| rapamycin, SDZ-RAD, | used for acute kidney rejection; |
| Everolimus | reduces rejection and graft |
| | vasculopathy following heart |
| | transplantation by inhibiting |
| | cell proliferation |
| 6-(3-dimethyl- | Immunosuppressing action useful |
| aminopropionyl) | also for treating autoimmune |
| forskolin | disease |
| 6-mercaptopurine | Used to treat Crohn's disease, |
| (6-MP) | inflammatory bowel disease and for |
| | organ transplant therapy |
| A-420983 | Lck-inhibitor |
| ABX-CBL (CBL-1) | Mouse monoclonal AB targeted against |
| | human T-cell, B cells, NK cells and |
| | monocytes, for treatment of steroid- |
| | resistant graft vs host |
| | diseases, potential use in treatment |
| | of inflammatory and autoimmune |
| | disorders |
| Alefacept (human | Knocks out causative memory T-lympho- |
| LFA-3 IgG1 fusion | cytes; Used to treat psoriasis, |
| protein) | a T-cell mediated inflammatory |
| | disorder |
| Antisense ICAM-1 | Mouse monoclonal AB blocks white |
| inhibitor (ISIS | blood cell adhesion to T-cell |
| 2302) Enlimomab, | surface molecule (ICAM-1r); treatment |
| BIRR1, | of kidney transplant rejection |
| Alicaforsen | |
| Antithymocyte | Anti-human thymocyte, immunoglobulin; |
| immunoglobulin | used in reversal of acute kidney |
| (ATGAM) | transplant rejection and will likely |
| | be used off-label for transplant |
| | induction therapy |
| Azathioprine | Treatment of rheumatoid arthritis and |
| | prevention of kidney transplant |
| | rejection, and other autoimmune or |
| | inflammatory disorders such as |
| | inflammatory bowel disease |
| Baohuoside-1 | Flavonoid; inhibits lymphocyte |
| | activation; Ma et al., Transplantation |
| | 78: 831-838, (2004) |
| basiliximab | Monoclonal AB that binds to receptor |
| | sites on T-cells, preventing |
| | activation by transplanted tissue |
| | (renal transplant) |
| BMS-279700 | Lck-inhibitor |
| BTI-322 | Mouse derived monoclonal AB targeted |
| | to CD2 receptor; used for prevention |
| | of first-time kidney rejection, and |
| | treatment of resistant rejection |
| Cladribine | Antimetabolite and immunosuppressive |
| | agent that is relatively selective for |
| | lymphocytes; used to treat lymphoid |
| | malignancies, e.g., hairycell leukemia |
| CP-690550 | JAK-3 inhibitor |
| Cyclophosphamide | Immunosuppressant for treatment of |
| (CTX) | arthritis and other auto-immune |
| | disorders and cancers |
| Cyclosporine | 11 amino acid cyclic peptide; blocks |
| (cyclosporin A, | helper T-cell, immunosuppressant |
| cyclosporin) | used in organ transplant therapy |
| | and other immune diseases |
| Daclizumab, HAT | Monoclonal AB inhibits binding of |
| (Humanized Anti- | IL-2 to IL-2 receptor by binding |
| Tac), SMART anti- | to IL-2 receptor; suppresses T-cell |
| Tac, anti-CD25, | activity against allografts (renal |
| and humanized | transplant) |
| anti-IL2-receptor | |
| Dexamethasone | An adrenocorticoid, effective |
| (Decadron, Dexone, | immunosuppressant in various |
| Dexasone) | disorders |
| DIAPEP-277 | Immunomodulatory properties |
| Dipeptide Boronic | Proteasome inhibitor. Wu et al., |
| Acid (DPBA) | Transplantation 78: 360-366, (2004) |
| Docosahexaenoic | Immunosuppressant that lowers the |
| acid (DHA) | proportion of T cells expressing |
| | CD4 or CD8, blocks antigen recog- |
| | nition process; Taku et al., Journal |
| | of Agricultural and Food Chemistry |
| | 48: 1047, (2000) |
| efalizumab | T-cell modulator that target T-cells |
| | through interactions with adhesion |
| | molecules on endothelial cell surface, |
| | target migration of T-cells into the |
| | skin and target activation of T-cells; |
| | Used to treat Psoriasis |
| Efomycine M | Leukocyte adhesion inhibitor, |
| | Anti-inflammatory |
| FTY720 (oral | Alters lymphocyte infiltration into |
| myriocin derivative) | grafted tissues; used for prevention |
| | of organ rejection in kidney |
| | transplants |
| Glatiramer acetate | Synthetic peptide copolymer, decoy |
| (co-polymer-1) | that mimics structure of myelin |
| | so immune cells bind Copaxone |
| | instead of myelin; for multiple |
| | sclerosis |
| Glial fibrillary | Possesses immunosuppressive |
| acidic protein | activities in diabetic animal models; |
| (GFAP) | Winer et al., Nature Medicine 9: |
| | 198, (2003) |
| Gusperimus (15- | Intravenous immunosuppressant; |
| deoxyspergualin) | suppresses production of cytotoxic |
| | T-cells, neutrophils and macrophages |
| HLA-B2702 peptide | Human peptide, blocks action of |
| | NK cells and T-cell mediated |
| | toxicities, used for prevention |
| | of first kidney allograft rejection |
| hu1124 (anti-CD11a) | Humanized monoclonal antibody; |
| | targets CD11a receptor on surface |
| | of T cells to selectively inhibit |
| | immune system rejection of |
| | transplanted organs |
| hOKT31γ(Ala—Ala) | non Fc-binding humanized anti |
| | CD3 antibody |
| Infliximab | Monoclonal AB, binds and inactivates |
| | human TNFalpha; used to treat |
| | Crohn's disease and rheumatoid |
| | arthritis |
| Interferon | Immunomodulatory properties |
| ISAtx247 | Used to treat autoimmune diseases |
| | such as rheumatoid arthritis and |
| | psoriasis |
| isotretinoin | Immunosuppressant, reduces ability |
| | of T cells to proliferate in |
| | response to immune challenge. |
| | Vergelli et al., Immunopharmacology, |
| | 31: 191, (1997) |
| L-683,742: also | Treatment of autoimmune diseases, |
| described as 31- | infectious diseases and/or |
| desmethoxy-31- | prevention of organ transplant |
| hydroxy-L-683,590 | rejections |
| Leflunomide (ARAVA) | Antiinflammatory agent |
| Medi-500 (T10B9) | Intravenous monoclonal AB that |
| | targets human T-cells; treats acute |
| | kidney rejection and graft-vs-host |
| | disease |
| Medi-507 | Intravenous humanized AB directed |
| | against CD2 T-cell; used to treat |
| | corticosteroidresistant graft vs |
| | host disease and prevention of kidney |
| | rejection |
| Methotrexate | Antimetabolite used to treat Crohn's |
| | disease, severe psoriasis, and |
| | adult rheumatoid arthritis (and as |
| | an anti-cancer drug) |
| Mitoxantrone | Antiproliferative effect on cellular |
| | immune system including T-cells, |
| | B-cells and macrophages; used to |
| | treat hormone-refractory prostate |
| | cancer, acute myelogenous leukemia |
| | and multiple sclerosis |
| mycophenolate | Proliferation of T and B lymphocytes |
| mofetil | by blocking the synthesis of |
| | purine nucleotides; used in organ |
| | transplant therapy and |
| | inflammatory bowel disease |
| OKT4A | Mouse monoclonal AB targeted against |
| | human CD4 T cell; used for |
| | prevention of kidney transplant |
| | rejection when used in combination |
| | with other immunosuppressant drugs |
| Muromonab-CD3 | Monoclonal AB that binds to receptor |
| | sites on T-cells, preventing |
| | activation by transplanted tissue |
| Prednisolone | Corticosteroid, suppresses inflam- |
| | mation associated with transplant |
| | rejection |
| Psora-4 | Kv1.3-blocker |
| Rifampicin | Antibiotic; has immunomodulatory |
| | properties |
| Rituximab | CD20 antibody |
| S100β | possesses immunosuppressive |
| | activities in diabetic animal models |
| Sirolimus, | Immunosuppressant and potent |
| Rapamycin | inhibitor of cytokine (e.g. IL-2)- |
| | dependent T-cell proliferation |
| | (kidney transplant) |
| Tacrolimus | Interferes with IL-2 TCR communication |
| (Prograf; FK-506) | |
| Triptolide | Small molecule; inhibits T-cell |
| | activation |

The combination therapy may comprise coadministration of the medicaments during the treatment period and/or separate administration of single medicaments during different time intervals in the treatment period.

The nucleic acids of the invention or fragments thereof, may further be useful in diagnostic applications, wherein the presence or amount of the nucleic acids or the proteins are to be assessed. Further antibodies that bind immunospecifically to the novel substances of the invention may be used in therapeutic or diagnostic methods.

For example, in one aspect, antibodies, which are specific for the proteins of the invention and homologous proteins, may be used directly as an effector/modulator, e.g. an antagonist or an agonist or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimeric single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof, which has immunogenic properties. Depending on the host species, various adjuvants may be used to increase immunological response. It is preferred that the peptides, fragments or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Köhler G. and Milstein C., (1975) Nature 256: 495-497; Kozbor D. et al., (1985) J. Immunol. Methods 81: 31-42; Cote R. J. et al., (1983) Proc. Natl. Acad. Sci. 80: 2026-2030; Cole S. P. et al., (1984) Mol. Cell Biol. 62: 109-120).

In addition, techniques developed for the production of ‘chimeric antibodies’, the splicing of mouse antibody genes to human antibody genes to obtain a molecule with appropriate antigen specificity and biological activity can be used (Morrison S. L. et al., (1984) Proc. Natl. Acad. Sci. 81: 6851-6855; Neuberger M. S. et al., (1984) Nature 312: 604-608; Takeda S. et al., (1985) Nature 314: 452-454). Alternatively, techniques described for the production of single chain antibodies may be adapted, using methods known in the art, to produce single chain antibodies specific for the proteins of the invention and homologous proteins. Antibodies with related specificity, but of distinct idiotypic composition, may be generated by chain shuffling from random combinatorial immunoglobulin libraries (Kang A. S. et al., (1991) Proc. Natl. Acad. Sci. 88: 11120-11123). Antibodies may also be produced by inducing in vivo production in the lymphocyte population or by screening recombinant immunoglobulin libraries or panels of highly specific binding reagents as disclosed in the literature (Orlandi R. et al., (1989) Proc. Natl. Acad. Sci. 86: 3833-3837; Winter G. and Milstein C., (1991) Nature 349: 293-299).

Antibody fragments, which contain specific binding sites for the proteins may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse W. D. et al., (1989) Science 246: 1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering protein epitopes are preferred, but a competitive binding assay may also be employed (Maddox, supra).

In another embodiment of the invention, the polynucleotides or fragments thereof or nucleic acid effector/modulator molecules such as antisense molecules, aptamers, RNAi molecules or ribozymes may be used for therapeutic purposes. In one aspect, aptamers, i.e. nucleic acid molecules, which are capable of binding to a protein of the invention and modulating its activity, may be generated by a screening and selection procedure involving the use of combinatorial nucleic acid libraries.

In a further aspect, antisense molecules may be used in situations in which it would be desirable to block the transcription of the mRNA. In particular, cells may be transformed with sequences complementary to polynucleotides encoding SF1-SF6 or homologous proteins. Thus, antisense molecules may be used to modulate/effect protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the proteins of the invention and homologous proteins. These techniques are described both in Sambrook et al. (supra) and in Ausubel et al. (supra). Genes encoding the proteins of the invention and homologous proteins can be turned off by transforming a cell or tissue with expression vectors, which express high levels of polynucleotides that encode the proteins of the invention and homologous proteins or fragments thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA or nucleic add analogues such as PNA to the control regions of the genes encoding SF1-SF6, i.e., the promoters, enhancers, and introns. Oligonucleotides derived from the transcription in initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee J. E. et al., (1994) Gene 149: 109-114; Huber B. E. and Carr B. I., (1994) Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the proteins of the invention and homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector/modulator molecules, e.g. antisense molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ and/or 3′ ends of the molecule or modifications in the nucleobase, sugar and/or phosphate moieties, e.g. the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of SF1-SF6 nucleic acids and the proteins and homologous nucleic acids or proteins, antibodies to the proteins of the invention and homologous proteins, mimetics, agonists, antagonists or inhibitors of the proteins of the invention and homologous proteins or nucleic acids. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, for example the SF1-SF6 nucleic acids or proteins or fragments thereof or antibodies, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide suffident levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 microg, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to the proteins may be used for the diagnosis of conditions or diseases characterized by or associated with over- or underexpression of the proteins of the invention and homologous proteins or in assays to monitor patients being treated with the proteins of the invention and homologous proteins, or effectors/modulators thereof, e.g. agonists, antagonists, or inhibitors. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules, which are known in the art may be used several of which are described above.

A variety of protocols including ELISA, RIA, and FACS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometric means. Quantities of protein expressed in control and disease, samples e.g. from biopsied tissues are compared with the standard values. Deviation between standard and subject values establishes the parameters for diagnosing disease.

In another embodiment of the invention, the polynucleotides specific for the SF1-SF6 proteins and homologous proteins may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with probes which are capable of detecting polynucleotide sequences, including genomic sequences, encoding the proteins of the invention and homologous proteins or closely related molecules, may be used to identify nucleic acid sequences which encode the respective protein. The hybridization probes of the subject invention may be DNA or RNA and are preferably derived from the nucleotide sequence of the polynucleotide encoding the proteins of the invention or from a genomic sequence including promoter, enhancer elements, and introns of the naturally occurring gene. Hybridization probes may be labeled by a variety of reporter groups, for example, radionuclides such as ³²P or ³⁵S or enzymatic labels, such as alkaline phosphatase coupled to the probe via avidin/biotin coupling systems, and the like.

Polynucleotide sequences specific for SF1-SF6 proteins or homologous nucleic acids may be used for the diagnosis of conditions or diseases, which are associated with the expression of the proteins. Examples of such diseases include the pancreatic diseases (e.g. diabetes), obesity, metabolic syndrome, and/or others. Polynucleotide sequences specific for the SF1-SF6 proteins may also be used to monitor the progress of patients receiving treatment for pancreatic diseases (e.g. diabetes), obesity, and/or metabolic syndrome. The polynucleotide sequences may be used qualitative or quantitative assays, e.g. in Southern or Northern analysis, dot blot or other membrane-based technologies; in PCR technologies; or in dip stick, pin, ELISA or chip assays utilizing fluids or tissues from patient biopsies to detect altered gene expression.

In a particular aspect, the SF1-SF6 nucleotide sequences may be useful in assays that detect activation or induction of various metabolic diseases or dysfunctions. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. The presence of altered levels of nucleotide sequences encoding the proteins of the invention and homologous proteins in the sample indicates the presence of the associated disease. Such assays may also be used to evaluate the efficacy of a particular therapeutic treatment regimen in animal studies, in clinical trials or in monitoring the treatment of an individual patient.

In order to provide a basis for the diagnosis of a disease associated with expression of the SF1-SF6 proteins, a normal or standard profile for expression is established. This may be accomplished by combining body fluids or cell extracts taken from normal subjects, either animal or human, with a sequence or a fragment thereof, which is specific for the nucleic acids encoding the proteins of the invention and homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to pancreatic diseases (e.g. diabetes), obesity, and/or metabolic syndrome, the presence of an unusual amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders.

Additional diagnostic uses for oligonucleotides designed from the sequences encoding the proteins of the invention and homologous proteins may involve the use of PCR. Such oligomers may be chemically synthesized, generated enzymatically or produced from a recombinant source. Oligomers will preferably consist of two nucleotide sequences, one with sense orientation (5prime.fwdarw.3prime) and another with antisense (3prime.rarw.5prime), employed under optimized conditions for identification of a specific gene or condition. The same two oligomers, nested sets of oligomers or even a degenerate pool of oligomers may be employed under less stringent conditions for detection and/or quantification of closely related DNA or RNA sequences.

In another embodiment of the invention, the nucleic acid sequences may also be used to generate hybridization probes, which are useful for mapping the naturally occurring genomic sequence. The sequences may be mapped to a particular chromosome or to a specific region of the chromosome using well known techniques. Such techniques include FISH, FACS or artificial chromosome constructions, such as yeast artificial chromosomes, bacterial artificial chromosomes, bacterial P1 constructions or single chromosome cDNA libraries as reviewed in Price C. M., (1993) Blood Rev. 7: 127-134, and Trask B. J., (1991) Trends Genet. 7: 149-154. FISH (as described in Verma R. S. and Babu A., (1989) Human Chromosomes: A Manual of Basic Techniques, Pergamon Press, New York, N.Y.). The results may be correlated with other physical chromosome mapping techniques and genetic map data. Examples of genetic map data can be found in the 1994 Genome Issue of Science (265: 1981f). Correlation between the location of the gene encoding the proteins of the invention on a physical chromosomal map and a specific disease or predisposition to a specific disease, may help to delimit the region of DNA associated with that genetic disease.

The nucleotide sequences of the subject invention may be used to detect differences in gene sequences between normal, carrier or affected individuals. An analysis of polymorphisms, e.g. single nucleotide polymorphisms may be carried out. Further, in situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti R. A. et al., (1988) Nature 336: 577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the proteins of the invention, their catalytic or immunogenic fragments or oligopeptides thereof, an in vitro model, a genetically altered cell or animal, can be used for screening libraries of compounds in any of a variety of drug screening techniques. One can identify effectors, e.g. receptors, enzymes, proteins, ligands, or substrates that bind to, modulate or mimic the action of one or more of the SF1-SF6 proteins of the invention. The protein or fragment thereof employed in such screening may be free in solution, affixed to a solid support, borne on a cell surface, or located intracellulary. The formation of binding complexes, between the SF1-SF6 proteins of the invention and the agent tested, may be measured. Agents could also, either directly or indirectly, influence the activity of the proteins of the invention.

In addition activity of the proteins of the invention against their physiological substrate(s) or derivatives thereof could be measured in cell-based or cell-free assays. Agents may also interfere with posttranslational modifications of the protein, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the proteins of the invention or, in a heterologous manner, of the proteins of the invention with other proteins, for example, but not exclusively, docking proteins, enzymes, receptors, or translation factors. Agents could also act on the physical interaction of the proteins of this invention with other proteins, which are required for protein function, for example, but not exclusively, their downstream signaling.

Methods for determining protein-protein interaction are well known in the art. For example binding of a fluorescently labeled peptide derived from the interacting protein to the SF1-SF6 protein of the invention, or vice versa, could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known in the art, for example but not exclusively AlphaScreen (PerkinElmer) or Scintillation Proximity Assays (SPA) by Amersham. Alternatively, the interaction of the SF1-SF6 proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI. In all cases the two or more binding partners can be different proteins with one being the protein of the invention, or in case of dimerization and/or oligomerization the protein of the invention itself.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to the proteins of the invention large numbers of different small test compounds, e.g. aptamers, peptides, low-molecular weight compounds etc., are provided or synthesized on a solid substrate, such as plastic pins or some other surface. The test compounds are reacted with the proteins or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support in another embodiment, one may use competitive drug screening assays in which neutralizing antibodies capable of binding the protein specifically compete with a test compound for binding the protein. In this manner, the antibodies can be used to detect the presence of any peptide, which shares one or more antigens determinants with the protein. Compounds that bind SF1-SF6 proteins, e.g. antibodies, are useful for the identification or enrichment of cells, which are positive for the expression of the proteins of the invention, from complex cell mixtures. Such cell populations are useful in transplantation, for experimental evaluation, and as source of lineage and cell specific products, including mRNA species useful in identifying genes specifically expressed in these cells, and as target for the identification of factors of molecules that can affect them. Cells expressing the protein of the invention or which have been treated with the protein of the invention are useful in transplantation to provide a recipient with pancreatic islet cells, including insulin producing beta cells; for drug screening; experimental models of islet differentiation and interaction with other cell types; in vitro screening assays to define growth and differentiation factors, and to additionally characterize genes involved in islet development and regulation; and the like. The native cells may be used for these purposes, or they may be genetically modified to provide altered capabilities. Cells from a regenerating pancreas, from embryonic foregut, stomach and duodenum, or other sources of pancreatic progenitor cells may be used as a starting population. The progenitor cells may be obtained from any mammalian species, e.g. equine, bovine, porcine, canine, feline, rodent, e.g. mice, rats, hamster, primate, etc. particularly human.

In another embodiment, in a high-throughput screening method, the cells are transfected with a DNA construct, e.g. a viral or non-viral vector containing a reporter gene, e.g. the lacZ gene or the GFP gene, under regulatory control of a promoter of a gene involved in for example beta-cell differentiation, e.g. a promoter of a gene stimulation beta-cell differentiation, preferably a Pax4 promoter. The transfected cells are divided into aliquots and each aliquot is contacted with a test substance, e.g., candidate 1, candidate 2 and candidate 3. The activity of the reporter gene corresponds to the capability of the test compound to induce beta-cell differentiation.

In a further embodiment, which may be combined with the high-throughput screening as described above, a medium throughput validation is carried out. Therein, the test compound is added to stem cells being cultivated and the insulin production is determined. Following an initial high throughput assay, such as the cell based assay outlined above where for example a Pax4 promoter is used as marker for beta-cell regeneration, the activity of candidate molecules to induce beta-cell differentiation is tested in a validation assay comprising adding said compounds to the culture media of the embryoid bodies. Differentiation into insulin-producing cells is then evaluated, e.g. by comparison to wild type and/or Pax4 expressing ES cells to assess the effectiveness of a compound.

The nucleic acids encoding the SF1-SF6 proteins of the invention can be used to generate transgenic cell lines and animals. These transgenic non-human animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test modulators of the protein of the invention. Misexpression (for example, overexpression or lack of expression) of the protein of the invention, particular feeding conditions, and/or administration of biologically active compounds can create models of metabolic disorders.

In one embodiment of the invention, such assays use mouse models of insulin resistance and/or diabetes, such as mice carrying gene knockouts in the leptin pathway (for example, ob (leptin) or db (leptin receptor) mice), as described above. In addition to testing the expression of the proteins of the invention in such mouse strains (see Examples), these mice could be used to test whether administration of a candidate modulator alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof. In tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lacZ or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transferred into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a nucleic acid molecule coding for a protein of the invention or a functional fragment thereof;
(b) a protein of the invention or a fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another effector/modulator against the nucleic acid of (a) or the polypeptide of (b), (e) or (f) and
(h) an anti-sense oligonucleotide of the nucleic acid of (a).

The kit may be used for diagnostic or therapeutic purposes or for screening applications as described above. The kit may further contain user instructions.

The Figures show:

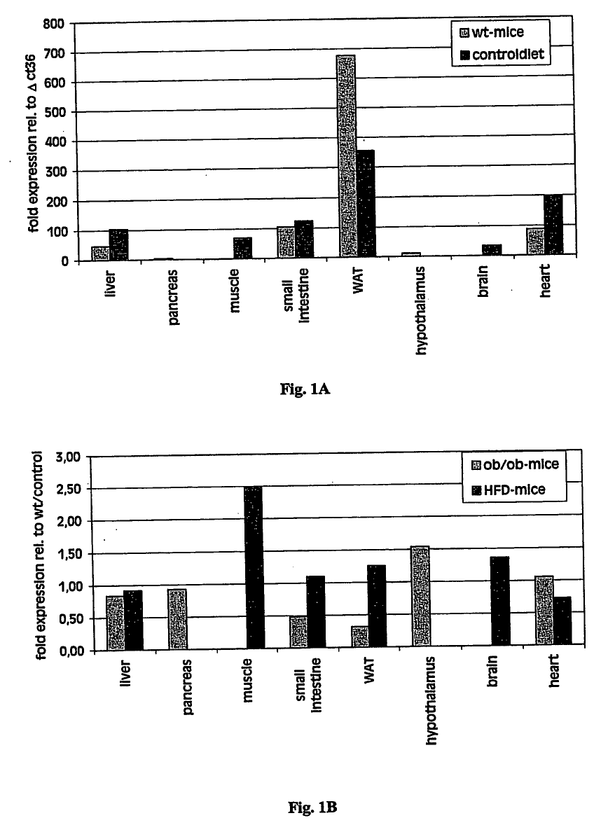

FIG. 1 shows the real-time PCR analysis of SF1 expression in mammalian (mouse) tissues.

FIG. 1A shows the analysis of SF1 expression in wild-type (referred to as wt-mice) and control diet (referred to as controldiet) mouse tissues.

FIG. 1B shows the analysis of SF1 expression in genetically obese mice (referred to as ob/ob-mice) compared to wild-type mice, and in mice fed with a high fat diet (referred to as HFD-mice) compared to mice fed with a control diet

FIG. 2 shows in situ hybridization results for the SF1 protein of the invention.

FIG. 2A shows the isolated gastrointestinal tract (lateral view) of a mouse embryo (day E11.5) stained with a whole-mount in situ hybridization method.

FIG. 2B shows the stomach (cross section) of a mouse embryo (day E11.5) stained with a whole-mount in situ hybridization method.

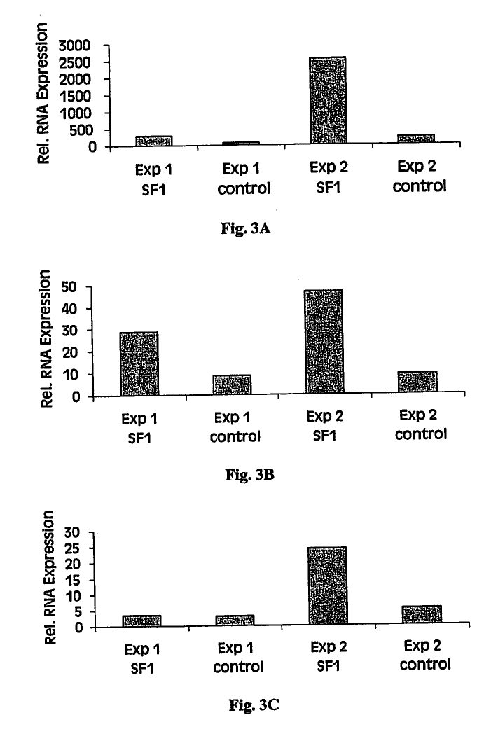

FIG. 3 shows the relative RNA expression of pancreatic genes in SF1 loss of function (LOF) zebrafish.

FIG. 3A shows insulin expression in SF1 LOF zebrafish.

FIG. 3B shows Pdx1 expression in SF1 LOF zebrafish.

FIG. 3C shows Pax4 expression in SF1 LOF zebrafish.

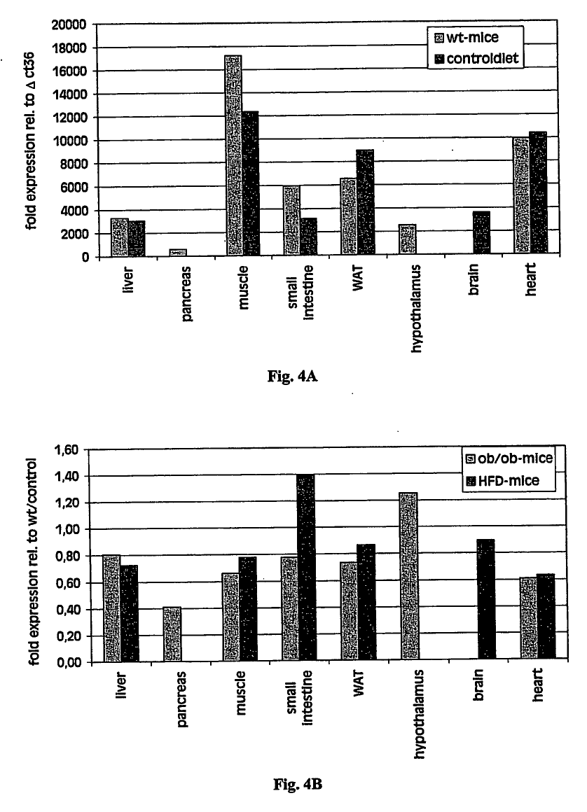

FIG. 4 shows the real-time PCR analysis of SF2 expression in mammalian (mouse) tissues.

FIG. 4A shows the analysis of SF2 expression in wild-type (referred to as wt-mice) and control diet (referred to as controldiet) mouse tissues.

FIG. 4B shows the analysis of SF2 expression in genetically obese mice (referred to as ob/ob-mice) compared to wild-type mice, and in mice fed with a high fat diet (referred to as HFD-mice) compared to mice fed with a control diet

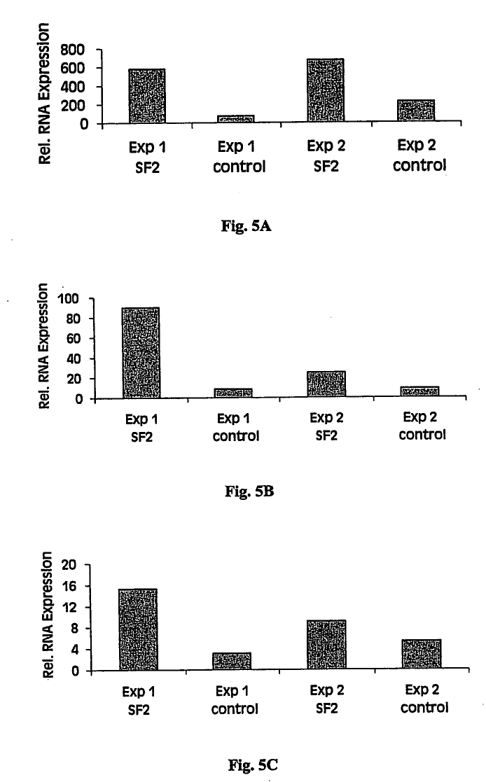

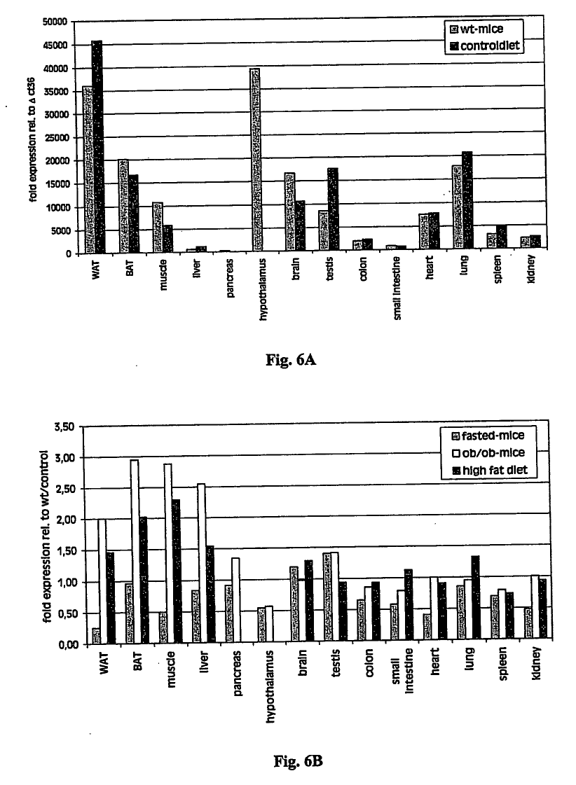

FIG. 5 shows the relative RNA expression of pancreatic genes in SF2 loss of function (LOF) zebrafish.

FIG. 5A shows insulin expression in SF2 LOF zebrafish.

FIG. 5B shows Pdx1 expression in SF2 LOF zebrafish.

FIG. 5C shows Pax4 expression in SF2 LOF zebrafish.

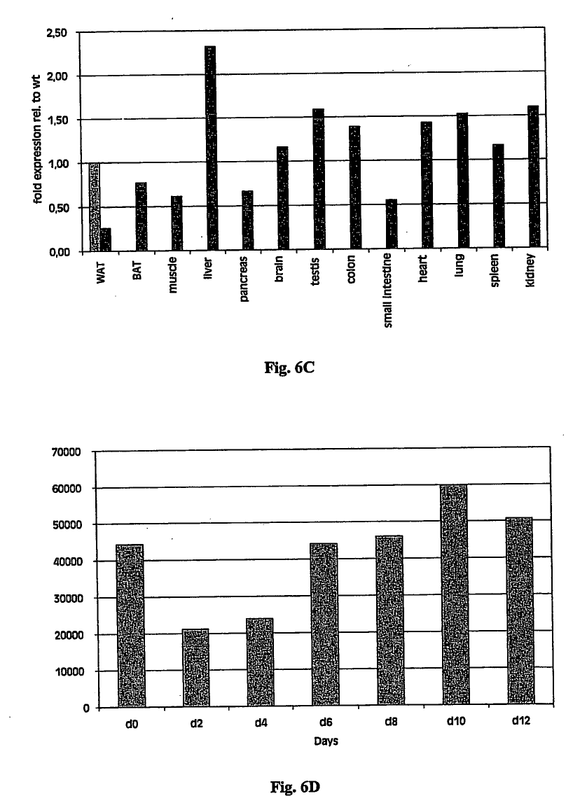

FIG. 6 shows the real-time PCR analysis of SF3 expression in mammalian (mouse) tissues.

FIG. 6A shows the analysis of SF3 expression wild-type (referred to as wt-mice) and control diet (referred to as controldiet) mouse tissues.

FIG. 6B shows the analysis of SF3 expression in genetically obese mice (referred to as ob/ob-mice) and fasted mice (referred to as fasted-mice) compared to wild-type mice and the expression in mice fed with a high fat diet (referred to as high fat diet) compared to mice fed with a control diet

FIG. 6C shows the analysis of SF3 expression in mouse tissues from non-obese-diabetic (referred to as NOD) mice compared to wild-type mice.

FIG. 6D shows the analysis of SF3 expression in mammalian fibroblast (3T3-L1) cells during the differentiation from preadipocytes to mature adipocytes.

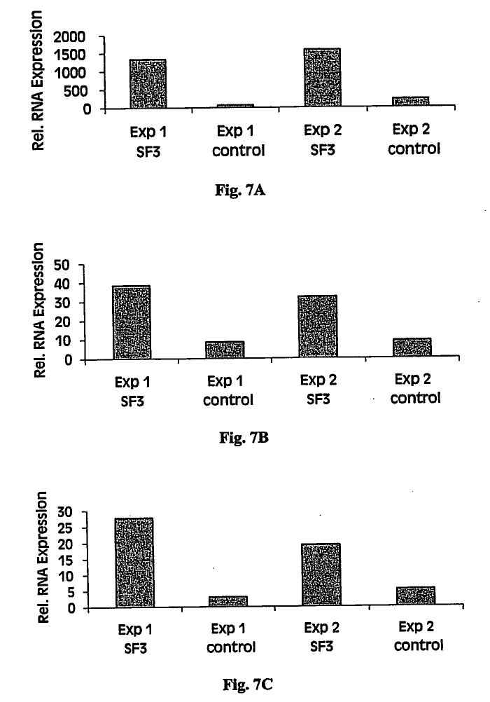

FIG. 7 shows the relative RNA expression of pancreatic genes in SF3 loss of function (LOF) zebrafish.

FIG. 7A shows insulin expression in SF3 LOF zebrafish.

FIG. 7B shows Pdx1 expression in SF3 LOF zebrafish.

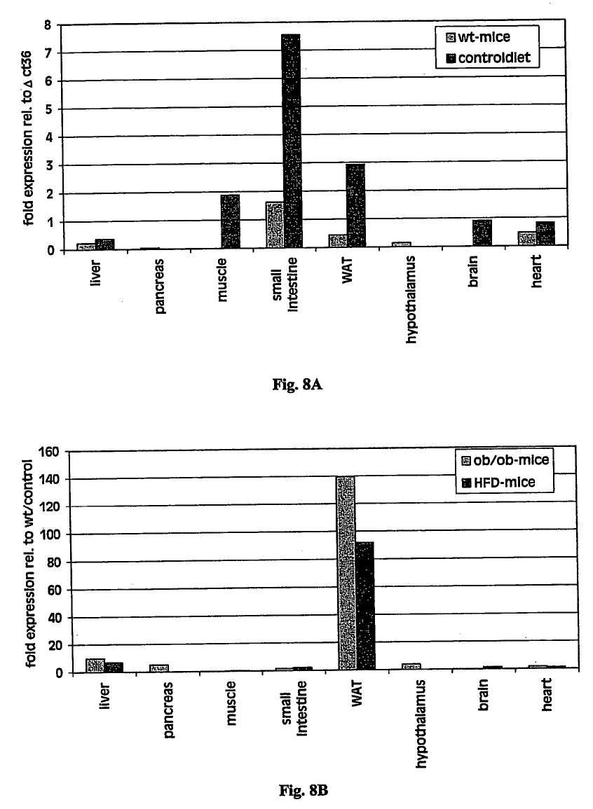

FIG. 8 shows the real-time PCR analysis of SF4 expression in mammalian (mouse) tissues.

FIG. 8A shows the analysis of SF4 expression in wild-type (referred to as wt-mice) and control diet (referred to as controldiet) mouse tissues.

FIG. 8B shows the analysis of SF4 expression in genetically obese mice (referred to as ob/ob-mice) compared to wild-type mice, and in mice fed with a is high fat diet (referred to as HFD-mice) compared to mice fed with a control diet

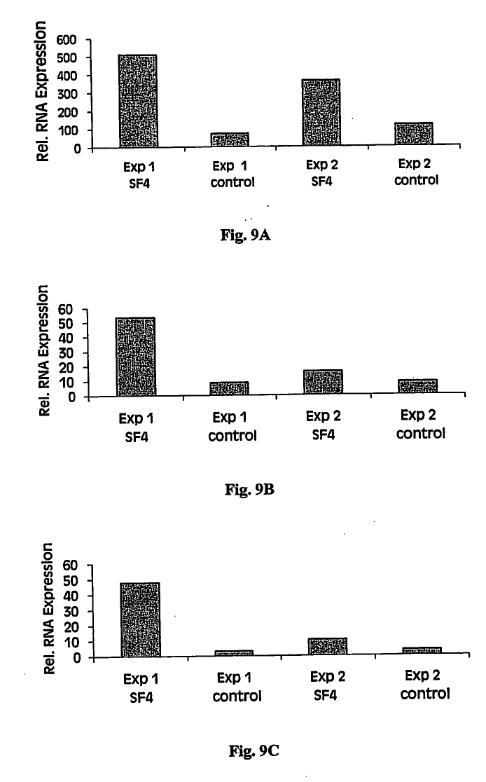

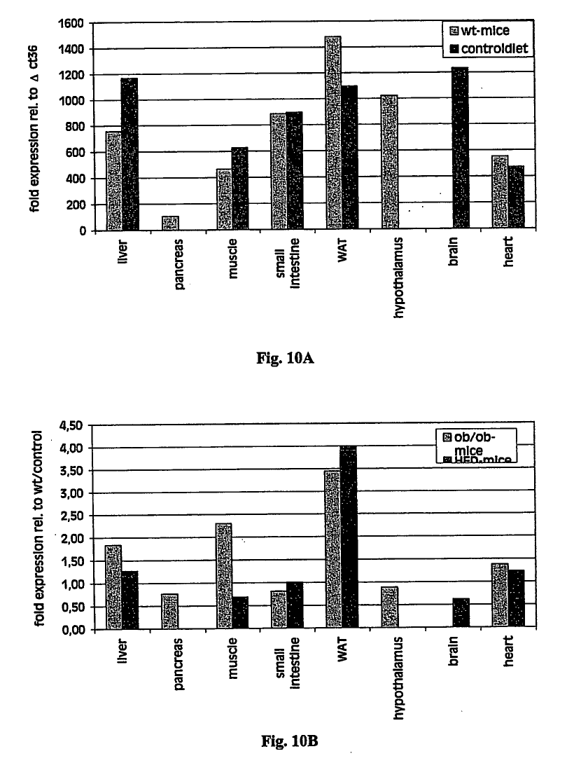

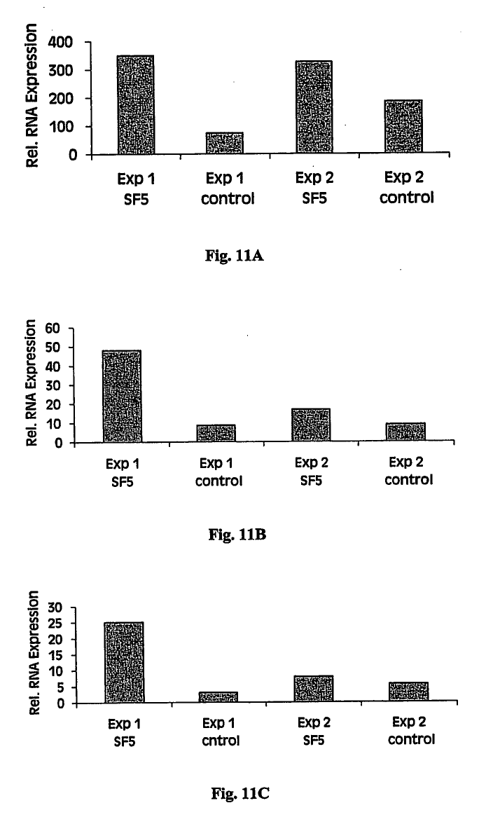

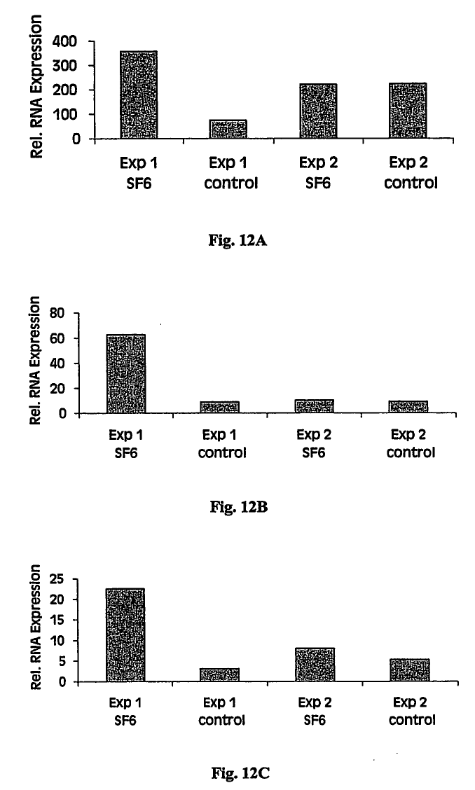

FIG. 9 shows the relative RNA expression of pancreatic genes in SF4 loss of function (LOF) zebrafish.

FIG. 9A shows insulin expression in SF4 LOF zebrafish.

FIG. 9B shows Pdx1 expression in SF4 LOF zebrafish.

FIG. 9C shows Pax4 expression in SF4 LOF zebrafish.

FIG. 10 shows the real-time PCR analysis of SF5 expression in mammalian (mouse) tissues.

FIG. 10A shows the analysis of SF5 expression in wild-type (referred to as wt-mice) and control diet (referred to as controldiet) mouse tissues.

FIG. 10B shows the analysis of SF5 expression in genetically obese mice (referred to as ob/ob-mice) compared to wild-type mice, and in mice fed with a high fat diet (referred to as HFD-mice) compared to mice fed with a control diet

FIG. 11 shows the relative RNA expression of pancreatic genes in SF5 loss of function (LOF) zebrafish.

FIG. 11A shows insulin expression in SF5 LOF zebrafish.

FIG. 11B shows Pdx1 expression in SF5 LOF zebrafish.

FIG. 11C shows Pax4 expression in SF5 LOF zebrafish.

FIG. 12 shows the relative RNA expression of pancreatic genes in SF6 loss of function (LOF) zebrafish.

FIG. 12A shows insulin expression in SF6 LOF zebrafish.

FIG. 12B shows Pdx1 expression in SF6 LOF zebrafish.

FIG. 12C shows Pax4 expression in SF6 LOF zebrafish.

FIG. 13 shows the in situ hybridization results for the SF3 protein of the invention.

FIG. 13A shows a cryosection of embryonic pancreas at day E17.5. Shown is a combined image of fluorescence and bright field picture (dark: in situ hybridization (referred to as ISH); red: insulin).

FIG. 13B shows a cryosection of embryonic pancreas at day E17.5. Shown is a combined image of fluorescence and bright field picture (dark: in situ hybridization (referred to as ISH); red: insulin).

The examples illustrate the invention:

### EXAMPLE 1

### Identification of Proteins Expressed in Pancreas

A screen for proteins expressed in developing mouse pancreas was carried out according to methods known by those skilled in the art (see, for example Pera E. M. and De Robertis E. M., (2000) Mech Dev 96(2): 183-195) with several modifications.

Expression cDNA Library:

During organogenesis, the pancreatic bud is surrounded and influenced by the associated mesenchyme. (see for example, Madsen O. D. et al., (1996) Eur. J. Biochem. 242: 435-445 and Slack, J. M., (1995) Development 121: 1569-1580). Recently, it was suggested, that white adipocytes origin directly from mesenchymal cells (Atanossova P. K, (2003) Folia Med. 45: 41-45). During embryogenesis, the innervation and vascularization of the pancreas can be observed. Therefore, the tissue used in the screen might have contained besides pancreatic cells some adipocyte precursors, blood vessels, as well as neuronal cells.

A mouse embryonic stage 9.5-15 pancreatic bud library was prepared in pCMVSPORT-6 vector using SUPERSCRIPT Plasmid System from Invitrogen according to the manufacturer's instructions. The non-amplified library was electroporated into MaxEff DH10B cells (Invitrogen).

Bacterial clones were picked with sterile toothpicks from agar plates and cultured in 96-deep-well microtiter plates in LB-ampicillin (see Sambrook et al., supra). Plasmid DNA was isolated using the BioRobot_{—}9600 apparatus according to the manufacturer's instructions (Qiagen; QIAprep(r) Turbo BioRobot Kit. Clones were partially sequenced from the 5′ end (SEQLAB, Goettingen).

### EXAMPLE 2

### Identification of the SF1-SF6 Homologous Nucleic Acid and Protein Sequences

The term “polynucleotide comprising the nucleotide sequence as shown in GenBank Accession number” relates to the expressible gene of the nucleotide sequences deposited under the corresponding GenBank Accession number. The term “GenBank Accession number” relates to NCBI GenBank database entries (Ref.: Benson D. A. et al., (2000) Nucleic Acids Res. 28: 15-18).

Sequences homologous to the mouse sequences were identified using the publicly available program BLASTP 2.2.3 of the non-redundant protein data base of the National Center for Biotechnology Information (NCBI) (see, Altschul S. F. et al., (1997) Nucleic Acids Res. 25: 3389-3402).

SF1, SF2, SF3, SF4, SF5, or SF6 proteins and nucleic acid molecules coding therefore are obtainable from insect or vertebrate species, e.g. mammals or fish. Particularly preferred are nucleic acid molecules and proteins encoded thereby comprising human SF1, SF2, SF3, SF4, SF5, or SF6, and mouse SF1, SF2, SF3, SF4, SF5, or SF6 sequences identified in the screen, as described in Table 2.

TABLE 2
Mammalian genes and proteins of the invention (SF1-SF6)

| | Genbank Accession Numbers | | | |
|---|---|---|---|---|
| | Mus musculus | | Homo sapiens | |
| Name | genes and proteins | | genes and proteins | |
| — | cDNA | Protein | cDNA | Protein |
| SF1 | AB050626 | BAC76038 | NM_022164 | NP_071447 |
| SF2 | XM_129394 | XP_129394 | NM_015999 | NP_057083 |
| SF3 | NM_009242 | NP_033268 | NM_003118 | NP_003109 |
| SF4 | NM_008108 | NP_032134 | NM_020634 | NP_065685 |
| SF5 | NM_029023 | NP_083299 | NM_021626 | NP_067639 |
| SF6 | NM_133883 | NP_598644 | NM_017825 | NP_060295 |

To identify possible zebrafish ortologues of human and mouse SF1, SF2, SF3, SF4, SF5, and SF6, sequence databases (NCBI non redundant protein database [ftp://ftp.ncbi.nih.gov/blast/db], EST section of NCBI Genbank (see Boguski et al., 1993, Nat Genet. 4: 332-333). dbEST-database for “expressed sequence tags”, and zebrafish genome draft assembly 2 [http://www.ensembl.org/Danio_rerio/]) were searched using the blastall programm (version 2.2.6, Altschul et al. 1997, Nucleic Acids Res. 25: 3389-3402). Starting from the blast hits candidate genes were assembled and translated as necessary using the programms genewise (version 2.2.0, see http://www.ebi.ac.uk/Wise2/), getorf, est2genome, and showseq (from the EMBOSS package version 2.7.1, see http://www.hgmp.mrc.ac.uk/Software/EMBOSS/). The resulting candidate protein sequences were compared to similar mouse and human proteins in multiple alignments made with the clustalw programm (version 1.83, see Thompson et al., 1994, Nucleic Acids Research, 22: 4673-4680) to verify the homology to mouse SF1, SF2, SF3, SF4, SF5, or SF6. If available from the assembly data, translation start sites were selected for antisense oligonucleotides targeting. Otherwise splice donor sites identified by alignment of zebrafish EST data or mouse protein data to zebrafish genomic sequence were used for antisense oligonucleotides targeting.

### EXAMPLE 3

### Loss-of-Function Experiments in Zebrafish

To study to effect of SF1, SF2, SF3, SF4, SF5, or SF6 on pancreatic function, several experiments were performed in zebrafish, which is a reliable model for vertebrate pancreatic development. Zebrafish were raised, maintained, and crossed as described (see, Westerfield M., (1995) The Zebrafish Book. Eugene, Oreg.: Univ. of Oregon Press). Staging was performed according to Kimmel C. B. et al., (1995) Dev Dyn 203: 253-310. Development of zebrafish embryos was carried out at 28° C. The age of embryos is indicated as hours post fertilization (hpf), the age of larvae as days post fertilization (dpf). Zebrafish carrying the transgene with insulin regulatory sequences linked to a fluroscent protein cDNA were used for the experiments. As control, progeny of crosses between AB and TL strain fish (Westerfield M. et al., supra; http://www.zfin.org) were used for injections.

SF1, SF2, SF3, SF4, SF5, or SF6 or control antisense oligonucleotides were injected into fertilized one-cell stage embryos as described (see, for example, Nasevicius & Ekker, 2000, Nat Genet 26: 216-220; Urtishak et al., 2003, Dev Dyn. 228: 405-413). Injected embryos were analysed at different stages of development or processed for quantitative RT-PCR at 48 hours post fertilization (hpf).

Loss-of-Function of SF1, SF2, SF3, SF4, SF5, and SF6 Stimulates Insulin, Pdx1, and Pax4 Expression in Zebrafish (Quantitative RT-PCR Analysis)

RNA was isolated from 20-30 fish embryos according to standard procedures, and quantitative RT-PCR (Taqman analysis) was performed according to standard procedures with primers specific for zebrafish insulin1 (GenBank Accession Number NP_{—}571131), zebrafish Pdx1 (GenBank Accession Number NP_{—}571518), zebrafish Pax 4 (Accession Number Icl| ctg9534 Zebrafish shotgun assembly V2) and 18S RNA (Accession Number D751553).

FIGS. 3, 5, 7, 9, 11, and 12 show the effect of loss-of-function of SF1, SF2, SF3, SF4, SF5, or SF6 on the expression levels of zebrafish insulin, pdx1, and pax4. The expression of insulin, Pdx4, and Pax4 is significantly enhanced upon inhibition of SF1, SF2, SF3, SF4, SF5, and SF6.

### EXAMPLE 4

### Quantitative Analysis of the Expression of Nucleic Acids in Mammalian (Mouse) Tissues

To analyse the expression of the SF1-SF5 mRNA disclosed in this invention in mammalian tissues, several mouse strains (preferably mice strains C57BI/6J, C57BI/6 ob/ob, C57BI/KS db/db, and Non-Obese-Diabetic (NOD) mice, which are standard model systems in obesity and diabetes research) were purchased from Harlan Winkelmann (33178 Borchen, Germany) and Taconic M & B (Germantown, N.Y. 12526, U.S.A.), respectively, and maintained under constant temperature (preferably 22° C.), 40 percent humidity and a light/dark cycle of preferably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum. (see, for example, Schnetzler et al., (1993) J Clin Invest 92: 272-280, Mizuno et al., (1996) Proc Natl Acad Sci USA 93: 3434-3438). In a further experiment wild-type (wt) mice were fed a control diet (preferably Altromin C1057 mod control, 4.5% crude fat) or high fat diet (preferably Altromin C1057mod. high fat, 23.5% crude fat). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 or cells were maintained as fibroblasts and differentiated into adipocytes as described in the prior art (e.g., Qiu. et al., J. Biol. Chem. 276:11988-95, 2001; Slieker et al., BBRC 251: 225-9, 1998). In brief, cells were plated in DMEM/10% FCS (Invitrogen, Karlsruhe, Germany) at 50,000 cells/well in duplicates in 6-well plastic dishes and cultured in a humidified atmosphere of 5% CO₂ at 37° C. At confluence (defined as day 0: d0) cells were transferred to serum-free (SF) medium, containing DMEM/Ham12 (3:1; Invitrogen), Fetuin (300 μg/ml; Sigma, Munich, Germany), transferrin (2 μg/ml; Sigma), pantothenate (17 μM; Sigma), Biotin (1 μM; Sigma), and EGF (0.8 nM; Hoffmann-La Roche, Basel, Switzerland). Differentiation was induced by adding dexamethasone (DEX; 1 μM; Sigma), 3-methyl-isobutyl-1-methylxanthine (MIX; 0.5 mM; Sigma), and bovine insulin (5 μg/ml; Invitrogen). Four days after confluence (d4), cells were kept in SF medium, containing bovine insulin (5 μg/ml) until differentiation was completed. At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days.

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art Total RNA was reverse transcribed (preferably using Superscript II RNaseH⁻ Reverse Transcriptase, from Invitrogen, Karlsruhe, Germany) and subjected to Taqman analysis preferably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

The following prime/probe pairs were used for the TaqMan analysis of the mouse SF1 sequence (GenBank Accession Number AB050626):

Mouse SF1 forward primer (Seq ID NO:1): 5′-CGT GCT GAC GAG TGT GCC-3′; mouse SF1 reverse primer (Seq ID NO:2): 5′- CGG TTG CAG AAG AGG TCA CAG-3′; mouse SF1 Taqman probe (Seq ID NO:3): (5/6-FAM)-TGC CCT ACC TGG GAG CCA CCT G-(5/6-TAMRA).

The following prime/probe pairs were used for the TaqMan analysis of the mouse SF2 sequence (GenBank Accession Number XM_{—}129394):

Mouse SF2 forward primer (Seq ID NO:4): 5′-GGC ACA GTG GGA CCG GT-3′; mouse SF2 reverse primer (Seq ID NO:5): 5′-AGC CAA GTC CCA GGA ACA CTC-3′; mouse SF2 Taqman probe (Seq ID NO:6): (5/6-FAM)-ACT CCC AAG CAC CGG CAG ACA AGA G-(5/6-TAMRA).

The following prime/probe pairs were used for the TaqMan analysis of the mouse SF3 sequence (GenBank Accession Number NM_{—}009242):

Mouse SF3 forward primer (Seq ID NO:7): 5′-CAC CTG GAC TAC ATC GGA CCA-3′; mouse SF3 reverse primer (Seq ID NO:8): 5′-GCA TGC GCA GAG GGA ATT-3′; mouse SF3 Taqman probe (Seq ID NO:9): (5/6-FAM)-CCC TGC CTG GAT TCC GAG CTG AC-(5/6-TAMRA).

The following prime/probe pairs were used for the TaqMan analysis of the mouse SF4 sequence (GenBank Accession Number NM_{—}008108):

Mouse SF4 forward primer (Seq ID NO:10): 5′-CCT MG GTG GGC AGA TTG CTT-3′; mouse SF4 reverse primer (Seq ID NO:11): 5′-GGT TGA ACT GGA GCT GAC CTT G-3′; mouse SF4 Taqman probe (Seq ID NO:12): (5/6-FAM)-CTG CGG TCT GTC CCT GGG CC-(5/6-TAMRA).

The following prime/probe pairs were used for the TaqMan analysis of the mouse SF5 sequence (GenBank Accession Number NM_{—}029023):

Mouse SF5 forward primer (Seq ID NO:13): 5′-AGC CCG GAG AAA GCT ATG GA-3′; mouse SF5 reverse primer (Seq ID NO:14): 5′-ACG TGG CGC TGA CAG AGA C-3′; mouse SF5 Taqman probe (Seq ID NO:15): (5/6-FAM)-TCG AGC CTC GAG TTC CTT CGG AGC-(5/6-TAMRA).

In the figures the relative RNA-expression is shown on the Y-axis. In FIGS. 1, 4, 6A-C, 8, and 10, the tissues tested are given on the X-axis. “WAT” refers to white adipose tissue, “BAT” refers to brown adipose tissue. In FIGS. 1, 4, 8, and 10, the panel of the wild type mice tissues comprises liver, pancreas, muscle, small intestine, WAT, hypothalamus, and heart, and the panel of the control diet-mice tissues comprises liver, muscle, small intestine, WAT, brain, and heart. In FIG. 6A the panel of the wild type mice tissues comprises WAT, BAT, muscle, liver, pancreas, hypothalamus, brain, testis, colon, small intestine, heart, lung, spleen, and kidney, and the panel of the control diet-mice tissues comprises WAT, BAT, muscle, liver, brain, testis, colon, small intestine, heart lung, spleen, and kidney. In FIG. 6D, the X-axis represents the time axis. “d0” refers to day 0 (start of the experiment), “d2”-“d12” refers to day 2-day 12 of adipocyte differentiation.

The function of the SF1-SF5 proteins of the invention in metabolism was further validated by analyzing the expression of the transcripts in different tissues. In one embodiment of this invention, mouse models of insulin resistance and/or diabetes were used, such as mice carrying gene knockouts in the leptin pathway (for example, ob/ob (leptin) or db/db (leptin receptor/ligand) mice) to study the expression of the proteins of the inversion. Such mice develop typical symptoms of diabetes, show hepatic lipid accumulation and frequently have increased plasma lipid levels (see Bruning J. C. et al, (1998) Mol. Cell. 2: 559-569).

Expression of the mRNAs encoding the proteins of the invention was also examined in susceptible wild type mice (for example, C57BI/6) that show symptoms of diabetes, lipid accumulation, and high plasma lipid levels, if fed a high fat diet. Expression profiling studies confirm the particular relevance of the proteins of the present invention as regulators of energy metabolism in mammals.

Taqman analysis revealed that SF1 is expressed in several mammalian tissues, showing highest level of expression in white adipose tissue (WAT), and lower but still robust levels in further tissues, e.g., heart, small intestine, liver, muscle, and brain, as shown in FIG. 1A. We found, for example, that the expression of SF1 is down-regulated in the WAT and small intestine of ob/ob mice compared to wild-type mice (see FIG. 1B). In wild type mice fed a high fat diet, the expression of SF1 is unregulated in the muscle. The ubiquitous and robust expression levels of SF1 suggest that it plays an essential role in cellular metabolism. The regulation of gene expression in different mouse models used to study metabolic disorders as described above suggests that it also might play a role in the regulation of energy homeostasis.

Taqman analysis revealed that SF2 is expressed in several mammalian tissues, showing highest level of expression in muscle and heart, and higher levels in further tissues, e.g. WAT, small intestine, liver, brain, and hypothalamus. Furthermore SF2 is expressed on lower but still robust levels in the pancreas as depicted in FIG. 4A. We found, for example, that the expression of SF2 is down-regulated in the pancreas of ob/ob mice compared to wild-type mice (see FIG. 4B). In wild type mice fed a high fat diet, the expression of SF2 is not regulated. The ubiquitous and robust expression levels of SF2 suggest that it plays an essential role in cellular metabolism. The regulation of gene expression in the mouse model for the metabolic syndrome as described above suggests that it also might play a role in the regulation of energy homeostasis.

Taqman analysis revealed that SF3 is expressed in several mammalian tissues, with highest expression levels in hypothalamus and white adipose tissue (WAT). In addition, SF3 is also highly expressed in brown adipose tissue (BAT), lung, brain, testis, muscle, and heart of wild type mice and control diet as depicted in FIG. 6A. Furthermore, SF3 is expressed on lower but still robust levels in spleen, kidney, colon, liver, small intestine, and pancreas. We found, for example, that the expression of SF3 is up-regulated in metabolic active tissues, including WAT, BAT, liver, and in muscle of genetically induced obese mice (ob/ob) compared to wild type mice. In addition the expression of SF3 is strongly down-regulated in WAT, and significantly down-regulated in muscle, heart and kidney of fasted wild type mice compared to wild-type mice fed a standard diet (see FIG. 6B). In one embodiment of the invention, expression of SF3 mRNA was also examined in susceptible wild type mice (for example, C57BI/6) that show symptoms of diabetes, lipid accumulation, and high plasma lipid levels, if fed a high fat diet. In those mice, the expression of SF3 is up-regulated in BAT and muscle when compared to control diet mice, supporting that SF3 is involved in the regulation of mammalian metabolism (see FIG. 6B). The expression of SF3 is significantly down-regulated in WAT and up-regulated in liver of non obese diabetic (NOD) mice, compared to wild type mice, as shown in FIG. 6C. We show in this invention (see FIG. 6D) that the SF3 mRNA is highly expressed during the differentiation into mature adipocyctes. Therefore, the SF3 protein might play an essential role in adipogenesis.

Taqman analysis revealed that the expression of SF4 is restricted to a few mammalian tissues, including small intestine and WAT as depicted in FIG. 8A. We found, for example, that the expression of SF4 is significantly up-regulated in metabolic active tissues (e.g. WAT and liver) of genetically induced obese mice (ob/ob) compared to wild type mice, and in wild type mice fed a high fat diet compared to mice fed a control diet (see FIG. 8B), supporting that SF4 is involved in the regulation of mammalian metabolism.

Taqman analysis revealed that SF5 is expressed in several mammalian tissues, showing highest level of expression in WAT, brain, hypothalamus, small intestine, and liver, and higher levels in further tissues, e.g. muscle and heart. Furthermore SF5 is expressed on lower but still robust levels in the pancreas as depicted in FIG. 10A. We found, for example, that the expression of SF5 is up-regulated in the WAT and muscle of ob/ob mice compared to wild-type mice (see FIG. 10B). In wild type mice fed a high fat diet, the expression of SF5 is up-regulated in WAT compared to mice fed a control diet. The ubiquitous and robust expression levels of SF5 suggest that it plays an essential role in cellular metabolism. The regulation of gene expression in different mouse models used to study metabolic disorders as described above suggests that it also might play a role in the regulation of energy homeostasis.

### EXAMPLE 5

### In Situ Hybridisations

Whole-mount and sectional in situ hybridizations were performed according to standard protocols as known to those skilled in the art and as described previously (for example, Pelton, R. W. et al., (1990) Development 110: 609-620; Belo, J. A. et al., (1997) Mech. Dev. 68: 45-57).

The nucleic acid sequence encoding the mouse SF1 protein is expressed in the stomach and the inner organs, with a weak expression in pancreas (see FIG. 2). The nucleic acid sequence encoding the mouse SF3 protein is expressed in the pancreatic tissue that surrounds the endocrine islet (see FIG. 13).

For the purpose of the present invention, it will be understood by the person having average skill in the art, that any combination of any feature mentioned throughout the specification is explicitly disclosed herewith.

All publications and patents mentioned in the above specification are herein incorporated by reference. Various modifications and variations of the described method and system of the invention will be apparent to those skilled in the art without departing from the scope and spirit of the invention. Although the invention has been described in connection with specific to preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising a SF1, SF2, SF3, SF4, SF5, or SF6 protein and/or a functional fragment thereof, a nucleic acid molecule encoding a SF1, SF2, SF3, SF4, SF5, or SF6 protein and/or a functional fragment thereof and/or an effector/modulator of said nucleic acid molecule and/or said protein or protein fragment.

2. The composition of claim 1, wherein the composition contains pharmaceutically acceptable carriers, diluents, and/or additives.

3. The composition of claim 1 or 2, wherein the nucleic acid molecule is a mammalian SF1, SF2, SF3, SF4, SF5, or SF6 nucleic acid, particularly encoding the human SF1, SF2, SF3, SF4, SF5, or SF6 polypeptide and/or a nucleic molecule, which is complementary thereto or a fragment thereof or a variant thereof.

4. The composition of any one of claims 1 to 3, wherein said nucleic acid molecule is selected from the group consisting of
(a) a nucleic acid molecule encoding a polypeptide as shown in Table 2, or an isoform, fragment or variant of the polypeptide as shown in Table 2;
(b) a nucleic acid molecule which comprises or is the nucleic acid molecule as shown in Table 2;
(c) a nucleic acid molecule being degenerate with as a result of the genetic code to the nucleic acid sequences as defined in (a) or (b),
(d) a nucleic acid molecule that hybridizes at 50° C. in a solution containing 1×SSC and 0.1% SDS to a nucleic acid molecule as defined in claim 3 or as defined in (a) to (c) and/or a nucleic acid molecule which is complementary thereto;
(e) a nucleic acid molecule that encodes a polypeptide which is at least 85%, preferably at least 90%, more preferably at least 95%, more preferably at least 98% and up to 99.6% identical to the human SF1, SF2, SF3, SF4, SF5, or SF6, as defined in claim 3 or to a polypeptide as defined in (a);
(f) a nucleic acid molecule that differs from the nucleic acid molecule of (a) to (e) by mutation and wherein said mutation causes an alteration, deletion, duplication or premature stop in the encoded polypeptide

5. The composition of any one of claims 14, wherein the nucleic acid molecule is a DNA molecule, particularly a cDNA or a genomic DNA.

6. The composition of any one of claims 1-5, wherein said nucleic acid encodes a polypeptide contributing to regulating the metabolism, in particular human metabolism.

7. The composition of any one of claims 1-6, wherein said nucleic acid molecule is a recombinant nucleic acid molecule.

8. The composition of any one of claims 1-7, wherein the nucleic acid molecule is a vector, particularly an expression vector.

9. The composition of any one of claims 1-6, wherein the polypeptide is a recombinant polypeptide.

10. The composition of claim 9, wherein said recombinant polypeptide is a fusion polypeptide.

11. The composition of any one of claims 1-8, wherein said nucleic acid molecule is selected from hybridization probes, primers and anti-sense oligonucleotides.

12. The composition of any one of claims 1-11 which is a diagnostic composition.

13. The composition of any one of claims 1-11 which is a therapeutic composition.

14. The composition of any one of claims 1-13 for the manufacture of an agent for detecting and/or verifying, for the treatment, alleviation and/or prevention of pancreatic diseases (e.g. diabetes such as insulin dependent diabetes mellitus and/or non insulin dependent diabetes mellitus and/or LADA), obesity, metabolic syndrome and/or other metabolic diseases or dysfunctions.

15. The composition of any one of claims 1-14 for the manufacture of an agent for the modulation of pancreatic development.

16. The composition of any one of claims 1-15 for the manufacture of an agent for the regeneration of pancreatic tissues or cells, particularly pancreatic beta cells.

17. The composition of any one of claims 1-16 for application in vivo.

18. The composition of any one of claims 1-16 for application in vitro.

19. The composition of any one of claims 1-18 in combination with a further pharmaceutical agent.

20. The composition of claim 19 wherein the further pharmaceutical agent is an agent suitable for the treatment or prevention of pancreatic diseases and/or obesity and/or metabolic syndrome.

21. The composition of claim 19 wherein the further pharmaceutical agent which has immunosuppressive activity.

22. Use of a SF1, SF2, SF3, SF4, SF5, or SF6 nucleic add molecule or a polypeptide encoded thereby or a fragment or a variant of said nucleic add molecule or said polypeptide and/or an effector/modulator of said nucleic acid or polypeptide for the manufacture of a medicament for the treatment of pancreatic diseases (e.g. diabetes such as insulin dependent diabetes mellitus and/or non insulin dependent diabetes mellitus and/or LADA), obesity, metabolic syndrome and/or other metabolic diseases or dysfunctions for controlling the function of a gene and/or a gene product which is influenced and/or modified by a SF1, SF2, SF3, SF4, SF5, or SF6 polypeptide.

23. Use of the SF1, SF2, SF3, SF4, SF5, or SF6 nucleic acid molecule or use of a polypeptide encoded thereby, or use of a fragment or a variant of said nucleic acid molecule or said polypeptide, or use of an effector/modulator of said nucleic acid molecule or said polypeptide for identifying substances capable of interacting with a SF1, SF2, SF3, SF4, SF5, or SF6 polypeptide in vitro and/or in vivo.

24. A non-human transgenic animal exhibiting a modified expression of a SF1, SF2, SF3, SF4, SF5, or SF6 polypeptide.

25. The animal of claim 24, wherein the expression of the SF1, SF2, SF3, SF4, SF5, or SF6 polypeptide is increased and/or reduced.

26. A recombinant host cell exhibiting a modified expression of a SF1, SF2, SF3, SF4, SF5, or SF6 polypeptide, or a recombinant host cell which comprises a nucleic acid molecule as defined in any one of claims 1 to 7.

27. The cell of claim 26 which is a human cell.

28. A method of identifying a (poly)peptide involved in the regulation of energy homeostasis and/or metabolism in a mammal comprising the steps of
(a) contacting a collection of (poly)peptides with a SF1, SF2, SF3, SF4, SF5, or SF6 homologous polypeptide or a fragment thereof under conditions that allow binding of said (poly)peptides;
(b) removing (poly)peptides which do not bind and
(c) identifying (poly)peptides that bind to said SF1, SF2, SF3, SF4, SF5, or SF6 homologous polypeptide.

29. A method of screening for an agent which effects/modulates the interaction of a SF1, SF2, SF3, SF4, SF5, or SF6 polypeptide with a binding target comprising the steps of
(a) incubating a mixture comprising
(aa) a SF1, SF2, SF3, SF4, SF5, or SF6 polypeptide or a fragment thereof;
(ab) a binding target/agent of said SF1, SF2, SF3, SF4, SF5, or SF6 polypeptide or fragment thereof; and
(ac) a candidate agent
under conditions whereby said polypeptide or fragment thereof specifically binds to said binding target at a reference affinity;
(b) detecting the binding affinity of said SF1, SF2, SF3, SF4, SF5, or SF6 polypeptide or fragment thereof to said binding target to determine an affinity for the agent, and
(c) determining a difference between affinity for the agent and reference affinity.

30. A method for screening for an agent, which effects/modulates the activity of a SF1, SF2, SF3, SF4, SF5, or SF6 polypeptide, comprising the steps of
(a) incubating a mixture comprising
(aa) a SF1, SF2, SF3, SF4, SF5, or SF6 polypeptide or a fragment thereof, and
(ab) a candidate agent
under conditions whereby said SF1, SF2, SF3, SF4, SF5, or SF6 polypeptide or fragment thereof exhibits a reference activity,
(b) detecting the activity of said SF1, SF2, SF3, SF4, SF5, or SF6 polypeptide or fragment thereof to determine an activity for the agent; and
(c) determining a difference between activity for the agent and reference activity.

31. A method of producing a composition comprising the (poly)peptide identified by the method of claim 28 or the agent identified by the method of claim 29 or 30 with a pharmaceutically acceptable carrier and/or diluent.

32. The method of claim 31 wherein said composition is a pharmaceutical composition for preventing, alleviating or treating of diseases and disorders, including pancreatic diseases (e.g. diabetes), obesity, and/or metabolic syndrome.

33. Use of a (poly)peptide as identified by the method of claim 28 or of an agent as identified by the method of claim 29 or 30 for the preparation of a pharmaceutical composition (i) for the treatment, alleviation and/or prevention of pancreatic diseases (e.g. diabetes), obesity, and/or metabolic syndrome, (ii) for the modulation of pancreatic development and/or (iii) for the regeneration of pancreatic cells or tissues.

34. Use of a nucleic acid molecule as defined in any one of claims 1 to 7 or 11 for the preparation of a medicament (i) for the treatment, alleviation and/or prevention of diseases or dysfunctions, including pancreatic diseases (e.g. diabetes), obesity, and/or metabolic syndrome, (ii) for the modulation of pancreatic development and/or (iii) for the regeneration of pancreatic cells or tissues.

35. Use of a polypeptide as defined in any one of claims 1 to 6, 9 or 10 for the preparation of a medicament (i) for the treatment, alleviation and/or prevention of pancreatic diseases (e.g. diabetes), obesity, and/or metabolic syndrome, (ii) for the modulation of pancreatic development and/or (iii) for the regeneration of pancreatic cells or tissues.

36. Use of a vector as defined in claim 8 for the preparation of a medicament (i) for the treatment, alleviation and/or prevention of pancreatic diseases (e.g. diabetes), obesity, and/or metabolic syndrome, (ii) for the modulation of pancreatic development and/or (iii) for the regeneration of pancreatic cells or tissues.

37. Use of a host cell as defined in claim 26 or 27 for the preparation of a medicament (i) for the treatment, alleviation and/or prevention of pancreatic diseases (e.g. diabetes), obesity, and/or metabolic syndrome, (ii) for the modulation of pancreatic development and/or (iii) for the regeneration of pancreatic cells or tissues.

38. Use of any one of claims 33-37 wherein the pharmaceutical composition comprises a further pharmaceutical agent.

39. Use of a SF1, SF2, SF3, SF4, SF5, or SF6 nucleic acid molecule or of a fragment thereof for the production of a non-human transgenic animal which over- or under-expresses the SF1, SF2, SF3, SF4, SF5, or SF6 gene product.

40. Kit comprising at least one of
(a) a SF1, SF2, SF3, SF4, SF5, or SF6 nucleic acid molecule or a functional fragment or an isoform thereof;
(b) a SF1, SF2, SF3, SF4, SF5, or SF6 amino acid molecule or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a), expressed by the vector of (c) or the host cell of (d);
(f) a fusion polypeptide encoded by the nucleic acid of (a);
(g) an antibody, an aptamer or another effector/modulator against the nucleic acid of (a) or the polypeptide of (b), (e), or (f) and/or
(h) an anti-sense oligonucleotide of the nucleic acid of (a).


SEQUENCE LISTING (39 sequences)

SEQ ID NO: 1 - DNA, 18 bp, Artificial Sequence
  Description of Artificial Sequence Synthetic primer
cgtgctgacgagtgtgcc

SEQ ID NO: 2 - DNA, 21 bp, Artificial Sequence
  Description of Artificial Sequence Synthetic primer
cggttgcagaagaggtcacag

SEQ ID NO: 3 - DNA, 22 bp, Artificial Sequence
  Description of Artificial Sequence Synthetic primer
tgccctacctgggagccacctg

SEQ ID NO: 4 - DNA, 17 bp, Artificial Sequence
  Description of Artificial Sequence Synthetic primer
ggcacagtgggaccggt

SEQ ID NO: 5 - DNA, 21 bp, Artificial Sequence
  Description of Artificial Sequence Synthetic primer
agccaagtcccaggaacactc

SEQ ID NO: 6 - DNA, 25 bp, Artificial Sequence
  Description of Artificial Sequence Synthetic primer
actcccaagcaccggcagacaagag

SEQ ID NO: 7 - DNA, 21 bp, Artificial Sequence
  Description of Artificial Sequence Synthetic primer
cacctggactacatcggacca

SEQ ID NO: 8 - DNA, 18 bp, Artificial Sequence
  Description of Artificial Sequence Synthetic primer
gcatgcgcagagggaatt

SEQ ID NO: 9 - DNA, 23 bp, Artificial Sequence
  Description of Artificial Sequence Synthetic primer
ccctgcctggattccgagctgac

SEQ ID NO: 10 - DNA, 21 bp, Artificial Sequence
  Description of Artificial Sequence Synthetic primer
cctaaggtgggcagattgctt

SEQ ID NO: 11 - DNA, 22 bp, Artificial Sequence
  Description of Artificial Sequence Synthetic primer
ggttgaactggagctgaccttg

SEQ ID NO: 12 - DNA, 20 bp, Artificial Sequence
  Description of Artificial Sequence Synthetic primer
ctgcggtctgtccctgggcc

SEQ ID NO: 13 - DNA, 20 bp, Artificial Sequence
  Description of Artificial Sequence Synthetic primer
agcccggagaaagctatgga

SEQ ID NO: 14 - DNA, 19 bp, Artificial Sequence
  Description of Artificial Sequence Synthetic primer
acgtggcgctgacagagac

SEQ ID NO: 15 - DNA, 24 bp, Artificial Sequence
  Description of Artificial Sequence Synthetic primer
tcgagcctcgagttccttcggagc

SEQ ID NO: 16 - DNA, 2177 bp, Homo sapiens
gcgggccagcctgggccccagcccacaccttcaccagggcccaggagccaccatgtggcgatgtccactggggctactgctgttgctgccgctggctggccacttggctctgggtgcccagcagggtcgtgggcgccgggagctagcaccgggtctgcacctgcggggcatccgggacgcgggaggccggtactgccaggagcaggacctgtgctgccgcggccgtgccgacgactgtgccctgccctacctgggcgccatctgttactgtgacctcttctgcaaccgcacggtctccgactgctgccctgacttctgggacttctgcctcggcgtgccacccccttttcccccgatccaaggatgtatgcatggaggtcgtatctatccagtcttgggaacgtactgggacaactgtaaccgttgcacctgccaggagaacaggcagtggcagtgtgaccaagaaccatgcctggtggatccagacatgatcaaagccatcaaccagggcaactatggctggcaggctgggaaccacagcgccttctggggcatgaccctggatgagggcattcgctaccgcctgggcaccatccgcccatcttcctcggtcatgaacatgcatgaaatttatacagtgctgaacccaggggaggtgcttcccacagccttcgaggcctctgagaagtggcccaacctgattcatgagcctcttgaccaaggcaactgtgcaggctcctgggccttctccacagcagctgtggcatccgatcgtgtctcaatccattctctgggacacatgacgcctgtcctgtcgccccagaacctgctgtcttgtgacacccaccagcagcagggctgccgcggtgggcgtctcgatggtgcctggtggttcctgcgtcgccgaggggtggtgtctgaccactgctaccccttctcgggccgtgaacgagacgaggctggccctgcgcccccctgtatgatgcacagccgagccatgggtcggggcaagcgccaggccactgcccactgccccaacagctatgttaataacaatgacatctaccaggtcactcctgtctaccgcctcggctccaacgacaaggagatcatgaaggagctgatggagaatggccctgtccaagccctcatggaggtgcatgaggacttcttcctatacaagggaggcatctacagccacacgccagtgagccttgggaggccagagagataccgccggcatgggacccactcagtcaagatcacaggatggggagaggagacgctgccagatggaaggacgctcaaatactggactgcggccaactcctggggcccagcctggggcgagaggggccacttccgcatcgtgcgcggcgtcaatgagtgcgacatcgagagcttcgtgctgggcgtctggggccgcgtgggcatggaggacatgggtcatcactgaggctgcgggcaccacgcggggtccggcctgggatccaggctaagggccggcggaagaggccccaatggggcggtgaccccagcctcgcccgacagagcccggggcgcaggcgggcgccagggcgctaatcccggcgcgggttccgctgacgcagcgccccgcctgggagccgcgggcaggcgagactggcggagcccccagacctcccagtggggacggggcagggcctggcctgggaagagcacagctgcagatcccaggcctctggcgcccccactcaagactaccaaagccaggacacctcaagtctccagccccactaccccaccccactcctgtattctttttttttttttttagacagggtcttgctccgttgcccaggttggagtgcagtggcccatcagggctcactgtaacctccgactcctgggttcaagtgaccctcccacctcagcctctcaagtagctgggactacaggtgcaccaccacacctggctaatttttgtattttttgtaaagaggggggtctcactgtgttgcccaggctggtctcgaactcctgggctcaagcggtccacctgcctccgcctcccaaagtgctgggattgcaggcatgagccactgcacccagccctgtattcttattcttcagatatttatttttcttttcactgttttaaaataaaaccaaagtattgataaaaaaaaaaaaaaaaa

SEQ ID NO: 17 - protein, 467 aa, Homo sapiens
MWRCPLGLLLLLPLAGHLALGAQQGRGRRELAPGLHLRGIRDAGGRYCQEQDLCCRGRADDCALPYLGAICYCDLFCNRTVSDCCPDFWDFCLGVPPPFPPIQGCMHGGRIYPVLGTYWDNCNRCTCQENRQWQCDQEPCLVDPDMIKAINQGNYGWQAGNHSAFWGMTLDEGIRYRLGTIRPSSSVMNMHEIYTVLNPGEVLPTAFEASEKWPNLIHEPLDQGNCAGSWAFSTAAVASDRVSIHSLGHMTPVLSPQNLLSCDTHQQQGCRGGRLDGAWWFLRRRGVVSDHCYPFSGRERDEAGPAPPCMMHSRAMGRGKRQATAHCPNSYVNNNDIYQVTPVYRLGSNDKEIMKELMENGPVQALMEVHEDFFLYKGGIYSHTPVSLGRPERYRRHGTHSVKITGWGEETLPDGRTLKYWTAANSWGPAWGERGHFRIVRGVNECDIESFVLGVWGRVGMEDMGHH

SEQ ID NO: 18 - DNA, 1938 bp, Mus musculus
cgagcttggaggcggaggcgcgcagggcagcctgggtccagcccacacccctcaccaggaggcaccatgtggggatgttggctggggctgctcttgctgctgctggctggccaggctgccctggaggcccggcggagtcgttggcgcagggagctggcgccagggctgcacctgcggggcatccgggacgccggtggcagatactgccaagagcaggacatgtgctgtcgaggccgtgctgacgagtgtgccctgccctacctgggagccacctgttactgtgacctcttctgcaaccgcaccgtctctgactgctgccccgacttctgggacttctgcctcgggattccaccccccttccctcccgtccaagggtgcatgcatgggggccggatctacccagtcttcggaacctactgggacaactgcaatcgatgcacctgccatgagggagggcattgggagtgcgaccaggagccgtgtctagtggacccagacatgattaaagccatcaaccgaggcaactacgggtggcaggctgggaaccacagtgccttctggggcatgaccctggatgagggcattcgctaccgcctgggcacaatccgcccatcctccactgtcatgaatatgaatgagatttatacggtgctgggccaaggggaagtgctacccactgcctttgaagcttcagagaagtggcccaacctgatccacgagccattggaccagggcaattgtgcaggttcctgggctttctccacagcagctgtcgcatctgatcgcgtctctatccattctttgggacacatgacacccatcctatcaccgcaaaacctgctgtcctgtgatacccaccaccagcagggctgccgaggtgggcgtcttgatggcgcttggtggttcctgcggcgccgcggggtggtgtctgacaactgttacccattctccggccgtgagcagaacgaggccagccccactcctcgttgtatgatgcacagccgcgccatggggcggggcaagcgccaagccacttcccgctgccccaatggtcaggttgattccaacgacatctaccaggtcacgcctgcctaccgcctgggctctgatgagaaggagatcatgaaggagctaatggaaaacggccctgttcaagcactcatggaagtacacgaggacttcttcttgtaccagcgtggcatctacagccacacacctgtaagccaggggaggccggagcagtaccgcagacatgggactcactcagtcaagatcactgggtggggagaagagacgctgccagacggaaggaccattaagtactggactgctgccaactcgtggggcccatggtggggtgaaaggggccacttccggatcgtgcgtggcaccaacgagtgcgacatcgagaccttcgtgctgggcgtctggggtcgcgtgggaatggaggacatggggcaccactgagtctcagccactaggcgaggtgggatccacagccacagaagaggccttgggggccatgcccgatgaagccttgtgtgcacttcgggaccaggtgctaatctctacagactcagatccgcgcgtgcgcgctaaggcagaatcccacctaggagacaaagatgcaccaggctggcggaagcccccagatatcacagccggaactgggaagggccctgtttggaaactgcagggagtatagacagattccaggtccctggtcagccaggccaagaccacaggagctaagacaccccaacctcatcaccctcctacccaccctctctctcatcttctttttgatgaattctgtccatctccctagcctccattttggttgtacctttccattctcagtactgctttcttattctttaaagatatttatttttcttttcattaaaataaaaccaaagtattgataaaaaaaaaaaa

SEQ ID NO: 19 - protein, 466 aa, Mus musculus
MWGCWLGLLLLLLAGQAALEARRSRWRRELAPGLHLRGIRDAGGRYCQEQDMCCRGRADECALPYLGATCYCDLFCNRTVSDCCPDFWDFCLGIPPPFPPVQGCMHGGRIYPVFGTYWDNCNRCTCHEGGHWECDQEPCLVDPDMIKAINRGNYGWQAGNHSAFWGMTLDEGIRYRLGTIRPSSTVMNMNEIYTVLGQGEVLPTAFEASEKWPNLIHEPLDQGNCAGSWAFSTAAVASDRVSIHSLGHMTPILSPQNLLSCDTHHQQGCRGGRLDGAWWFLRRRGVVSDNCYPFSGREQNEASPTPRCMMHSRAMGRGKRQATSRCPNGQVDSNDIYQVTPAYRLGSDEKEIMKELMENGPVQALMEVHEDFFLYQRGIYSHTPVSQGRPEQYRRHGTHSVKITGWGEETLPDGRTIKYWTAANSWGPWWGERGHFRIVRGTNECDIETFVLGVWGRVGMEDMGHH

SEQ ID NO: 20 - DNA, 2108 bp, Homo sapiens
ggcgctgaagatcggggccgctcggccgcaggccgcctccagcgccgcgggatgtagcgcgggggaccgcggcccccagcagagcccgcctgcccggcttgtctaccatcagagggagatctctgccccctggggctgagagaccccaacctttccccaagctgaagctgcagggtattgaggtaccagccagatgtcttcccacaaaggatctgtggtggcacaggggaatggggctcctgccagtaacagggaagctgacacggtggaactggctgaactgggacccctgctagaagagaagggcaaacgggtaatcgccaacccacccaaagctgaagaagagcaaacatgcccagtgccccaggaagaagaggaggaggtgcgggtactgacacttcccctgcaagcccaccacgccatggagaagatggaagagtttgtgtacaaggtctgggagggacgttggagggtcatcccatatgatgtgctccctgactggctaaaggacaacgactatctgctacatggtcatagacctcccatgccctcctttcgggcttgcttcaagagcatcttccgcattcatacagaaactggcaacatctggacccatctgcttggtttcgtgctgtttctctttttgggaatcttgaccatgctcagaccaaatatgtacttcatggcccctctacaggagaaggtggtttttgggatgttctttttgggtgcagtgctctgcctcagcttctcctggctctttcacaccgtctattgtcattcagagaaagtctctcggactttttccaaactggactattcagggattgctcttctaattatggggagctttgtcccctggctctattattccttctactgctccccacagccacggctcatctacctctccatcgtctgtgtcctgggcatttctgccatcattgtggcgcagtgggaccggtttgccactcctaagcaccggcagacaagagcaggcgtgttcctgggacttggcttgagtggcgtcgtgcccaccatgcactttactatcgctgagggctttgtcaaggccaccacagtgggccagatgggctggttcttcctcatggctgtgatgtacatcactggagctggcctttatgctgctcgaattcctgagcgcttctttcctggaaaatttgacatatggttccagtctcatcagattttccatgtcctggtggtggcagcagcctttgtccacttctatggagtctccaaccttcaggaattccgttacggcctagaaggcggctgtactgatgacacccttctctgagccttcccacctgcggggtggaggaggaacttcccaagtgcttttaaaaataacttctttgctgaagtgagaggaagagtctgagttgtctgtttctagaagaaacctcttagagaattcagtaccaaccaagcttcagcccactttcacacccactgggcaataaactttccatttccattctcctagctggggatggggcatggtcaaacttagccatcccctcctcagcaaggcatctaccggcccctcacagagacagtactttgaaactcatgttgagattttaccctctcctccaaccattttgggaaaattatggactgggactcttcagaaattctgtcttttcttctggaagaaaatgtccctcccttacccccatccttaactttgtatcctggcttataacaggccatccatttttgtagcacacttttcaaaaacaattatataccctggtcccatctttctagggcctggatctgcttatagagcaggaagaataaagccaccaacttttacctagcccggctaatcatggaagtgtgtccaggcttcaagtaacttgagttttaattttttttttttcttggcagagtaatgtaaaatttaaatggggaaagatatttaatatttaatactaagctttaaaaagaaacctgctatcattgctatgtatcttgatgcaaagactatgatgttaataaaagaaagtacagaagagacttggcattcaaagatttcaaaaaaaaaaaaaaaaaaaaaaaaaaaaaaaaaaaaaaaa

SEQ ID NO: 21 - protein, 375 aa, Homo sapiens
MSSHKGSVVAQGNGAPASNREADTVELAELGPLLEEKGKRVIANPPKAEEEQTCPVPQEEEEEVRVLTLPLQAHHAMEKMEEFVYKVWEGRWRVIPYDVLPDWLKDNDYLLHGHRPPMPSFRACFKSIFRIHTETGNIWTHLLGFVLFLFLGILTMLRPNMYFMAPLQEKVVFGMFFLGAVLCLSFSWLFHTVYCHSEKVSRTFSKLDYSGIALLIMGSFVPWLYYSFYCSPQPRLIYLSIVCVLGISAIIVAQWDRFATPKHRQTRAGVFLGLGLSGVVPTMHFTIAEGFVKATTVGQMGWFFLMAVMYITGAGLYAARIPERFFPGKFDIWFQSHQIFHVLVVAAAFVHFYGVSNLQEFRYGLEGGCTDDTLL

SEQ ID NO: 22 - DNA, 2083 bp, Mus musculus
cggagcgggtgaggtagcgccgggaaggtgacgcggctgcggaggtgacgccgtgcggtcgcgcggcccttccggcgcgggcagggcgctgaggatcgggcgcccctcggccgcaggccgcctccagtgccgcgggatgtagcgcccgggcccgcggcccccagcagaggcagccagccaggcctgtccaccatcacaggaagatctctgccctctggggctgagagaccccaacctttcttcaagctgaagctgccaggcactgaggtaccagccagatgtcttcccacaaaggctctgccggggcacaaggcaatggggctccttctggtaacagagaagctgacacagtggagctggctgagctggggcccctgctggaggagaagggcaagcgggcagccagcagcccagccaaggctgaggaagatcaagcatgcccggtgcctcaggaagaggaggaggaggtgcgggtgctgacgcttcctctgcaagcccaccatgccatggagaagatggaggagttcgtgtataaggtctgggagggacgttggagagtcatcccgtatgatgtgcttcctgactggctgaaagacaacgactacctgctacatggccacagaccacctatgccctcctttcgggcttgcttcaagagcatcttccgcatccacacagagactggcaacatctggacacatctgcttggttttgtgctatttctctttctgggaatcttgacgatgctgagaccaaatatgtacttcatggctcccctgcaggagaaggtggtcttcgggatgttcttcctgggcgcggtgctctgcctcagtttctcctggctcttccacactgtctactgtcattcagagaaggtctctcggactttttccaaactggactattcagggattgctctactgattatggggagcttcgttccctggctctattactccttctactgctccccacagccgcggctcatctacctctccatcgtctgtgtcctgggcatctctgccatcattgtggcacagtgggaccggtttgccactcccaagcaccggcagacaagagcaggagtgttcctgggacttggcttgagtggtgttgtacccaccatgcactttactatcgctgagggctttgtcaaggccaccacggtgggccagatgggctggttcttcctcatggctgtgatgtacatcaccggcgccggcctgtatgctgctcggattcctgagcgcttcttccctggaaaatttgacatctggttccagtctcatcagattttccacgtcctggtggtggcagcagctttcgtccacttctatggtgtgtccaaccttcaggaattccgttatggcctagaaggtggctgtaccgacgactcccttctctgagcctccccacctgaggggtggaggagggacttcccaagtgcttttaaaaataacttctttgctgaagtatgaggaagagtctgagttgtctgtttctagaagaaatctcttagagaagtcagtaccagtgacatgtcagctcactgtcccagcctccgtccgggcagtacacttcccatgtgcattcccggcagtcacctcggcaaggcaactgctgctccttcacagataggacgtgggagctcatgttgagattttacccattcctccagccattttgggaaaaaaaaaattatgaactgggattcttcagaaattatgttttgttttttcttgaagaaaatatcccccccttacccccgtccttactttgtaacctggctgataacgggccatccatttttgtagcacacttttcagaaacaattataacctggtcccatctttctaggacaaacaaagccacgccttgatgtagcccggctaatcatggaagtacgtccaggcttcaaataacttgagtttaaatccttttttttttttttcttggcagagtaatgtaaaattaaaatggggaagatatttaatatttaatactaagctttaaaaagaaacctgctatcactgctatgtatcttgatgcaaagactatggtgataataaaagaaagtatagac

SEQ ID NO: 23 - protein, 375 aa, Mus musculus
MSSHKGSAGAQGNGAPSGNREADTVELAELGPLLEEKGKRAASSPAKAEEDQACPVPQEEEEEVRVLTLPLQAHHAMEKMEEFVYKVWEGRWRVIPYDVLPDWLKDNDYLLHGHRPPMPSFRACFKSIFRIHTETGNIWTHLLGFVLFLFLGILTMLRPNMYFMAPLQEKVVFGMFFLGAVLCLSFSWLFHTVYCHSEKVSRTFSKLDYSGIALLIMGSFVPWLYYSFYCSPQPRLIYLSIVCVLGISAIIVAQWDRFATPKHRQTRAGVFLGLGLSGVVPTMHFTIAEGFVKATTVGQMGWFFLMAVMYITGAGLYAARIPERFFPGKFDIWFQSHQIFHVLVVAAAFVHFYGVSNLQEFRYGLEGGCTDDSLL

SEQ ID NO: 24 - DNA, 2133 bp, Homo sapiens
cgggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaatccaccaaccagaacgtggtttgcctgaggctgtaactgagagaaagattctggggctgtcttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctcccctttgttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatctttcaaaataaaaagtaatgacttagaaactgaaaaaaaaaaaaaaaaaaaaaaaaaaaaaa

SEQ ID NO: 25 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 26 - DNA, 2079 bp, Mus musculus
gcattcctgcagcccttcagaccgccagaactcttctgccgcctgcctgcctgcctgcctgcctgcctgtgccgagagttcccagcatcatgagggcctggatcttctttctcctttgcctggccgggagggccctggcagcccctcagcagactgaagttgctgaggagatagtggaggaggaaaccgtggtggaggagacaggggtacctgtgggtgccaacccagtccaggtggaaatgggagaatttgaggacggtgcagaggaaacggtcgaggaggtggtggctgacaacccctgccagaaccatcattgcaaacatggcaaggtgtgtgagctggacgagagcaacacccccatgtgtgtgtgccaggaccccaccagctgccctgctcccattggcgagtttgagaaggtatgcagcaatgacaacaagaccttcgactcttcctgccacttctttgccaccaagtgcaccctggagggcaccaagaagggccacaagctccacctggactacatcggaccatgcaaatacatcgccccctgcctggattccgagctgaccgaattccctctgcgcatgcgtgactggctcaaaaatgtcctggtcaccttgtacgagagagatgagggcaacaacctcctcactgagaagcagaagctgcgtgtgaagaagatccatgagaatgagaagcgcctggaggctggagaccaccccgtggagctgttggcccgagactttgagaagaactacaatatgtacatcttccctgtccactggcagtttggccagctggatcagcaccctattgatgggtacctgtcccacactgagctggccccactgcgtgctcccctcatccccatggaacattgcaccacacgtttctttgagacctgtgacctagacaacgacaagtacattgccctggaggaatgggccggctgctttggcatcaaggagcaggacatcaacaaggatctggtgatctaagttcacgcctcctgctgcagtcctgaactctctccctctgatgtgtcacccctcccattacccccttgtttaaaatgtttggatggttggctgttccgcctggggataaggtgctaacatagatttaactgaatacattaacggtgctaaaaaaaaaaaaaaaacaaagtaagaaagaaactagaacccaagtcacagcattttcccacataactctgaggccatggcccatccacagcctcctggtcccctgcactacccagtgtctcactggctgtgttggaaacggagttgcataagctcaccgtccacaagcacgagatatctctagctttcatttcaattttgcatttgactcttaacactcacccagactctgtgcttatttcattttgggggatgtgggctttttcccctggtggtttggagttaggcagagggaagttacagacacaggtacaaaatttgggtaaagatactgtgagacctgaggacccaccagtcagaacccacatggcaagtcttagtagcctaggtcaaggaaagacagaataatccagagctgtggcacacatgacagactcccagcagcccgggaccttgctgtcttctcgactcttcgggcgtttctttccatgtttggctgttggttttagttttggtgagccatgggtgggccagaacatcactcaactgcaattgggctttcaggttcttgccgggagctctaggcactgggaggctgtttcaggaaagtgagactcaagaggaagacagaaaaggttgtaacgtagaggaagtgagactggtgaattggtttgatttttttcacatctagatggctgtcataaagtttctagcatgttccccctcacctctccccaccccctgccacttgaaaccttctactaatcaagagaaacttccaagccaacggaatggtcagatctcacaggctgagaaattgttcccctccaagcatttcatgaaaaagctgcttctcattaaccatgcaaactctcacagcgatgtgaagagcttgacaagtctttcaaaataaaaagtaacaacttagaaacgg

SEQ ID NO: 27 - protein, 302 aa, Mus musculus
MRAWIFFLLCLAGRALAAPQQTEVAEEIVEEETVVEETGVPVGANPVQVEMGEFEDGAEETVEEVVADNPCQNHHCKHGKVCELDESNTPMCVCQDPTSCPAPIGEFEKVCSNDNKTFDSSCHFFATKCTLEGTKKGHKLHLDYIGPCKYIAPCLDSELTEFPLRMRDWLKNVLVTLYERDEGNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALEEWAGCFGIKEQDINKDLVI

SEQ ID NO: 28 - DNA, 1224 bp, Homo sapiens
ggagctctccccggtctgacagccactccagaggccatgcttcgtttcttgccagatttggctttcagcttcctgttaattctggctttgggccaggcagtccaatttcaagaatatgtctttctccaatttctgggcttagataaggcgccttcaccccagaagttccaacctgtgccttatatcttgaagaaaattttccaggatcgcgaggcagcagcgaccactggggtctcccgagacttatgctacgtaaaggagctgggcgtccgcgggaatgtacttcgctttctcccagaccaaggtttctttctttacccaaagaaaatttcccaagcttcctcctgcctgcagaagctcctctactttaacctgtctgccatcaaagaaagggaacagttgacattggcccagctgggcctggacttggggcccaattcttactataacctgggaccagagctggaactggctctgttcctggttcaggagcctcatgtgtggggccagaccacccctaagccaggtaaaatgtttgtgttgcggtcagtcccatggccacaaggtgctgttcacttcaacctgctggatgtagctaaggattggaatgacaacccccggaaaaatttcgggttattcctggagatactggtcaaagaagatagagactcaggggtgaattttcagcctgaagacacctgtgccagactaagatgctcccttcatgcttccctgctggtggtgactctcaaccctgatcagtgccacccttctcggaaaaggagagcagccatccctgtccccaagctttcttgtaagaacctctgccaccgtcaccagctattcattaacttccgggacctgggttggcacaagtggatcattgcccccaaggggttcatggcaaattactgccatggagagtgtcccttctcactgaccatctctctcaacagctccaattatgctttcatgcaagccctgatgcatgccgttgacccagagatcccccaggctgtgtgtatccccaccaagctgtctcccatttccatgctctaccaggacaataatgacaatgtcattctacgacattatgaagacatggtagtcgatgaatgtgggtgtgggtaggatgtcagaaatgggaatagaaggagtgttcttagggtaaatcttttaataaaactacctatctggtttatgaccacttagatcgaaatgtca

SEQ ID NO: 29 - protein, 364 aa, Homo sapiens
MLRFLPDLAFSFLLILALGQAVQFQEYVFLQFLGLDKAPSPQKFQPVPYILKKIFQDREAAATTGVSRDLCYVKELGVRGNVLRFLPDQGFFLYPKKISQASSCLQKLLYFNLSAIKEREQLTLAQLGLDLGPNSYYNLGPELELALFLVQEPHVWGQTTPKPGKMFVLRSVPWPQGAVHFNLLDVAKDWNDNPRKNFGLFLEILVKEDRDSGVNFQPEDTCARLRCSLHASLLVVTLNPDQCHPSRKRRAAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 30 - DNA, 1280 bp, Mus musculus
tgaggggctgagaagagagcaattcacacttgattagctcccaggctcctgaattgagcagaggaggctagaccgctgagctgcgcaccccagaggctgctctaccctggctcagacgaccatgcagccttatcaacggcttctggcgcttggcttccttctgttaaccctgccctggggccagacatccgagtttcaagactctgaccttttgcagtttctgggattagagaaagcgccttcacctcacaggttccaacctgtgcctcgcgtcttaaggaaaatcatccgggctcgagaagccgctgcagccagtggggcctcgcaggacttatgctacgtgaaggagctgggtgttcgtgggaacctgcttcagcttctcccagaccagggttttttccttaatacacagaaacctttccaagatggctcctgtctccagaaggtcctctattttaacttgtctgccatcaaagaaaaggcaaagttgaccatggcccagctgactctagacttggggcccaggtcctactataacctgcgaccagagctggtggttgctctgtctgtggttcaggaccggggcgtgtgggggcgatcccaccctaaggtgggcagattgctttttctgcggtctgtccctgggcctcaaggtcagctccagttcaacctgcagggtgcgcttaaggattggagcagcaaccgactgaagaatttggacttacacttagagattttggtcaaagaggacagatactccagggtaactgtccagcccgagaacccctgtgacccgctgctccgctctctacatgcctcgctgctggtggtaaccctcaatcctaaacactgtcatccttcttccagaaaaaggagggcggccatctctgtccccaagggtttctgtaggaacttctgccaccgtcatcagctgttcatcaacttccaggacctgggttggcacaagtgggtcatcgcccctaaggggttcatggcaaattactgtcatggagagtgccccttctcaatgaccacgtatttaaatagttccaattatgctttcatgcaggctctgatgcatatggctgaccccaaggtccccaaggctgtctgtgtccccaccaagctctcgcccatctccatgctctatcaggatagtgataagaacgtcattctccgacattatgaagacatggtagtcgatgagtgtgggtgtgggtagtctcgggactaggctaggagtgtgcttagggtaaatcctttaataaaactaccacccc

SEQ ID NO: 31 - protein, 366 aa, Mus musculus
MQPYQRLLALGFLLLTLPWGQTSEFQDSDLLQFLGLEKAPSPHRFQPVPRVLRKIIRAREAAAASGASQDLCYVKELGVRGNLLQLLPDQGFFLNTQKPFQDGSCLQKVLYFNLSAIKEKAKLTMAQLTLDLGPRSYYNLRPELVVALSVVQDRGVWGRSHPKVGRLLFLRSVPGPQGQLQFNLQGALKDWSSNRLKNLDLHLEILVKEDRYSRVTVQPENPCDPLLRSLHASLLVVTLNPKHCHPSSRKRRAAISVPKGFCRNFCHRHQLFINFQDLGWHKWVIAPKGFMANYCHGECPFSMTTYLNSSNYAFMQALMHMADPKVPKAVCVPTKLSPISMLYQDSDKNVILRHYEDMVVDECGCG

SEQ ID NO: 32 - DNA, 1921 bp, Homo sapiens
cctgttgctgatgctgccgtgcggtacttgtcatggagctggcactgcggcgctctcccgtcccgcggtggttgctgctgctgccgctgctgctgggcctgaacgcaggagctgtcattgactggcccacagaggagggcaaggaagtatgggattatgtgacggtccgcaaggatgcctacatgttctggtggctctattatgccaccaactcctgcaagaacttctcagaactgcccctggtcatgtggcttcagggcggtccaggcggttctagcactggatttggaaactttgaggaaattgggccccttgacagtgatctcaaaccacggaaaaccacctggctccaggctgccagtctcctatttgtggataatcccgtgggcactgggttcagttatgtgaatggtagtggtgcctatgccaaggacctggctatggtggcttcagacatgatggttctcctgaagaccttcttcagttgccacaaagaattccagacagttccattctacattttctcagagtcctatggaggaaaaatggcagctggcattggtctagagctttataaggccattcagcgagggaccatcaagtgcaactttgcgggggttgccttgggtgattcctggatctcccccgtggattcggtgctctcctggggaccttacctgtacagcatgtctcttctcgaagacaaaggtctggcagaggtgtctaaggttgcagagcaagtactgaatgccgtaaataaggggctctacagagaggccacagagctgtgggggaaagcagaaatgatcattgaacagaacacagatggggtgaacttctataacatcttaactaaaagcactcccacgtctacaatggagtcgagtctagaattcacacagagccacctagtttgtctttgtcagcgccacgtgagacacctacaacgagatgccttaagccagctcatgaatggccccatcagaaagaagctcaaaattattcctgaggatcaatcctggggaggccaggctaccaacgtctttgtgaacatggaggaggacttcatgaagccagtcattagcattgtggacgagttgctggaggcagggatcaacgtgacggtgtataatggacagctggatctcatcgtagataccatgggtcaggaggcctgggtgcggaaactgaagtggccagaactgcctaaattcagtcagctgaagtggaaggccctgtacagtgaccctaaatctttggaaacatctgcttttgtcaagtcctacaagaaccttgctttctactggattctgaaagctggtcatatggttccttctgaccaaggggacatggctctgaagatgatgagactggtgactcagcaagaataggatggatggggctggagatgagctggtttggccttggggcacagagctgagctgaggccgctgaagctgtaggaagcgccattcttccctgtatctaactggggctgtgatcaagaaggttctgaccagcttctgcagaggataaaatcattgtctctggaggcaatttggaaattatttctgcttcttaaaaaaacctaagattttttaaaaaattgatttgttttgatcaaaataaaggatgataatagatattattttttcttatgacagaagcaaatgatgtgatttatagaaaaactgggaaatacaggtacccaaagagtaaatcaacatctgtatacccccttcccaggggtaagcactgttaccaatttagcatatgtccttgcagaatttttttttctatatatacatatatattttttaccaaaatgaatcattactctatgttgttttactatttgtttgacatatcagtatatctgaaacaccttttcatgtcaataaatgttcttctctaacattaa

SEQ ID NO: 33 - protein, 452 aa, Homo sapiens
MELALRRSPVPRWLLLLPLLLGLNAGAVIDWPTEEGKEVWDYVTVRKDAYMFWWLYYATNSCKNFSELPLVMWLQGGPGGSSTGFGNFEEIGPLDSDLKPRKTTWLQAASLLFVDNPVGTGFSYVNGSGAYAKDLAMVASDMMVLLKTFFSCHKEFQTVPFYIFSESYGGKMAAGIGLELYKAIQRGTIKCNFAGVALGDSWISPVDSVLSWGPYLYSMSLLEDKGLAEVSKVAEQVLNAVNKGLYREATELWGKAEMIIEQNTDGVNFYNILTKSTPTSTMESSLEFTQSHLVCLCQRHVRHLQRDALSQLMNGPIRKKLKIIPEDQSWGGQATNVFVNMEEDFMKPVISIVDELLEAGINVTVYNGQLDLIVDTMGQEAWVRKLKWPELPKFSQLKWKALYSDPKSLETSAFVKSYKNLAFYWILKAGHMVPSDQGDMALKMMRLVTQQE

SEQ ID NO: 34 - DNA, 2078 bp, Mus musculus
cacggcgtcacgcgatggcctttggctcctgcctgttgctgatgttgcggcggggttttcatcatggagctctcgcggcggatctgtctcgtgcaactgtggctgctgctcctatcgttcttactgggcttcagcgcgggatctgccatccactggcgggaacccgaaggcaaggaagtatgggattatgtgactgtccgaaaggatgcccacatgttctggtggctctattatgccaccaacccttgcaagaacttttcagagctgcccctggtcatgtggcttcagggtggtccgggtggttctagcactggatttggaaactttgaggaaattggccctcttgacacccaactcaagcctcgaaataccacctggctgcagtgggccagtctcctgtttgtggataatcccgtgggcacgggcttcagctacgtcaacacaacagatgcctacgcaaaggacctggacacggtggcttccgacatgatggttctcctgaaatccttctttgattgccataaagaattccagacggttccattctacattttctcagaatcctacggaggaaagatggctgctggcatcagtgtagaactttacaaggctgttcagcaagggaccattaagtgcaacttttctggggttgctttgggtgactcctggatctcccccgtggattcagtgctgtcctggggaccttacctgtatagtatgtctctccttgataatcaaggcttggccgaggtgtccgacattgcagagcaagtcctcgatgctgtaaacaagggcttctacaaggaggccactcagctgtgggggaaagcagaaatgatcattgaaaagaacaccgacggggtaaacttctataacatcttaactaaaagcagcccggagaaagctatggaatcgagcctcgagttccttcggagccccttagttcgtctctgtcagcgccacgtgagacacctgcaaggagacgccttaagtcaactcatgaacggccccatcaaaaagaagctcaaaattatccctgaggatatctcctggggagcccaggcatcttacgtcttcctaagcatggaaggggacttcatgaagcctgccatcgacgttgtggataagttgctggcagctggggtcaatgtgaccgtgtacaacggacagctggatctcattgtggacaccataggtcaggagtcctgggttcagaagctcaagtggccacagctgtccaaattcaatcagctaaaatggaaggccctgtacaccgatcccaagtcttcagaaacagctgcgttcgtcaagtcctatgagaacctagccttctactggatcctaaaggccggtcacatggttccttctgaccaaggggagatggccctgaagatgatgaagctggtgaccaagcaggagtagctgagctggctggccctggaggcgctaagagcagagcccaggatgcaggtgctaatgtctatccctggtgctcttctcccccgctctgccatgggatatgactctgggagcacctgctctctcccgtaccaaaagactgtggccttctgtgtctacttagaaatcagttctgcttcccaaagagtatttaaaaccagactcatttaatcaaagtgaagggttgcaatcgattggtctcttactacaaaagcagttgatagcacatgtaaatcccagcacttgagaggtagaagaagcaagaggatggatgagaggttcaaacgcatccgcagctacgtcgaaagttcaaaagcagcctatgccaaacagggagagtccctgtcccccacccccacccccaaaaaagagcaaaagcaaaccacatgatttataggcaaaagcagtgggagagacaaagaaaatacttaaacacccactgagctgccaactaggtgtctctgactacgggtcttttatttgctacatatatatttttacaaaaatgaaatccatattgtacgctattttgctgtctgcttcgttcccatgtcaacatgacctgcacttcttttcccatcaataaatgtgttgtgatatttttaaaaaaaaaaaaaaaa

SEQ ID NO: 35 - protein, 452 aa, Mus musculus
MELSRRICLVQLWLLLLSFLLGFSAGSAIHWREPEGKEVWDYVTVRKDAHMFWWLYYATNPCKNFSELPLVMWLQGGPGGSSTGFGNFEEIGPLDTQLKPRNTTWLQWASLLFVDNPVGTGFSYVNTTDAYAKDLDTVASDMMVLLKSFFDCHKEFQTVPFYIFSESYGGKMAAGISVELYKAVQQGTIKCNFSGVALGDSWISPVDSVLSWGPYLYSMSLLDNQGLAEVSDIAEQVLDAVNKGFYKEATQLWGKAEMIIEKNTDGVNFYNILTKSSPEKAMESSLEFLRSPLVRLCQRHVRHLQGDALSQLMNGPIKKKLKIIPEDISWGAQASYVFLSMEGDFMKPAIDVVDKLLAAGVNVTVYNGQLDLIVDTIGQESWVQKLKWPQLSKFNQLKWKALYTDPKSSETAAFVKSYENLAFYWILKAGHMVPSDQGEMALKMMKLVTKQE

SEQ ID NO: 36 - DNA, 1691 bp, Homo sapiens
ggcgagcagtctgcgcgcggatggccgcagcggcgatggcggcagcggcaggtggaggggctggcgcggcccgctccctctcgcgcttccgaggctgcctggctggtgcgctgctcggggactgcgtgggctccttctacgaggcccacgacaccgtcgacctgacgtcagtcctgcgtcatgtccagagtctggagccggaccccggcacgcccgggagtgagcggacagaagccttgtactacacagatgacacagccatggccagggccctggtgcagtccctgctagccaaggaggcctttgacgaggtggacatggctcacagatttgctcaggagtacaagaaagaccctgacaggggctatggtgctggagtagtcactgtcttcaagaagctcctgaaccccaaatgtcgcgatgtctttgagcctgcccgggcccagtttaacgggaaaggctcctatggcaatggaggtgccatgcgggtggctggcatctccctggcctatagcagtgtccaggatgtgcagaagtttgcccggctctcggcccagctgacacacgcctcctccctgggttacaatggcgccatcctgcaggccctggctgtgcacctggccttgcagggcgagtcttccagcgagcactttctcaagcaactcctgggccacatggaggatctggagggtgatgcccagtccgtcttggatgccagggagttgggcatggaggagcgtccatactccagccgcctgaagaagattggagagcttctagaccaggcatcggtgaccagggaggaagtggtgtctgagctagggaatggcattgctgcctttgagtcggtacccaccgccatctactgcttcctacgctgcatggagccagaccctgagatcccttctgccttcaatagcctccaaaggactctcatttattccatctcacttggtggggacacagacaccattgccaccatggctggggccattgctggtgcctactatgggatggatcaggtgccagagagctggcagcaaagctgtgaaggctacgaggagacagacatcctggcccaaagcctgcaccgtgtcttccagaagagttgatgagggctacagctgttggggctctgccaggtcccctgggaccaactacagctccaatcagaaaccctgcgcttccttgagtgtggcttcccacttttcctgcattgtggagctgactgagtacaccggtgaggctggggtctctgcaggggaggtcactggaacagcgagcaagggactggtgcctcgctggtgctgggtctctggtttgctgcagagccgtaggacactcctggctcctcagtaggacagacagacgcaggcgggtttattttggaggggtacttgtggcattttcctgtattgtcttggacatgggatgtggggaggtggaaatgatgagcagtagcatcatttctccctgttgggttttagccagtttgccagcaagcgcatcctagcagggtccccgagcagcaggttgtgtggatgaagggacaggcacttgcatccagctgatctaggtcacacctggctcttggctgccatgtggcttattaacagcttccagtggaagtcgcaataaacagtttttggtaaatctcaaaaaaaaaaaaaaaaaaaaaaaaaaaaaaaaaa

SEQ ID NO: 37 - protein, 363 aa, Homo sapiens
MAAAAMAAAAGGGAGAARSLSRFRGCLAGALLGDCVGSFYEAHDTVDLTSVLRHVQSLEPDPGTPGSERTEALYYTDDTAMARALVQSLLAKEAFDEVDMAHRFAQEYKKDPDRGYGAGVVTVFKKLLNPKCRDVFEPARAQFNGKGSYGNGGAMRVAGISLAYSSVQDVQKFARLSAQLTHASSLGYNGAILQALAVHLALQGESSSEHFLKQLLGHMEDLEGDAQSVLDARELGMEERPYSSRLKKIGELLDQASVTREEVVSELGNGIAAFESVPTAIYCFLRCMEPDPEIPSAFNSLQRTLIYSISLGGDTDTIATMAGAIAGAYYGMDQVPESWQQSCEGYEETDILAQSLHRVFQKS

SEQ ID NO: 38 - DNA, 1368 bp, Mus musculus
cgatgccggaagtggcgactagtccgcgcgcggatggcggtggctgcggcggcagcagctacagcgatgtcggcggcggggggcggcggggcaagtgcggcccgctccatctcgcgcttccgaggttgcctggcgggcgcgctgctgggagattgcgtgggcgctgtctacgaggcacacgataccgtcagcctggcatcagtcctgagtcacgtcgagagcctggagccggacccgggcacgccgggcagcgcgcggacagagacactgtactacacagatgacactgccatgaccagggccctggtacagtccctgctggccaaggaggccttcgacgaggtggacatggctcacaggtttgcccaggaatacaagaaggaccctgacagagggtatggggccggagtcatcactgtcttcaagaaactcctgaatcccaagtgccgtgatgtctatgagcctgcccgggcccagttcaacgggaagggttcctatggcaatgggggtgccatgcgggtagcaggcatctcgctggcctatagcagtgtccaagatgtacagaagtttgcccggctctcagcccagctgacccacgcctcttccctgggctataacggtgccatcttgcaggccctggctgtgcaccttgctctgcagggtgtatcatccagtgagcacttcctcgagcagcttctgggccacatggaggagctggaaggtgatgcccagtcagtcttggacgccaaggagttgggtatggaggagcgtccgtactccagcaggctgaagaaggtcggagagctgctggaccaggacgtggtgagccgagaggaagtggtgtccgagctagggaatggcattgccgcctttgaatctgtgcccaccgccatctactgcttcctgcgctgcatggagcctcaccctgagatcccctccaccttcaacagtctccagaggactctcatctactccatctcacttggtggggacacagacaccatagccaccatggctggggccattgctggagcttactatgggatggaacaggtgccggagagctggcagcaaagttgtgaaggctttgaggagacagacgtcctggcccagagcctgcaccgagtcttccaggagagctcgtaagggccgcagcttcggtgggcttgtcagacccatcaggaccaaggacaactcagattggactccacagttcccttaggttgccgcacctgccctcaatcctagagggaagactgaaggtacccttggggctggaatcttgagacagcctgtggaacagagaggagctggtgccttcttggggctgcagctctggcttgctgcagggctgtatgacacccaggg

SEQ ID NO: 39 - protein, 370 aa, Mus musculus
MAVAAAAAATAMSAAGGGGASAARSISRFRGCLAGALLGDCVGAVYEAHDTVSLASVLSHVESLEPDPGTPGSARTETLYYTDDTAMTRALVQSLLAKEAFDEVDMAHRFAQEYKKDPDRGYGAGVITVFKKLLNPKCRDVYEPARAQFNGKGSYGNGGAMRVAGISLAYSSVQDVQKFARLSAQLTHASSLGYNGAILQALAVHLALQGVSSSEHFLEQLLGHMEELEGDAQSVLDAKELGMEERPYSSRLKKVGELLDQDVVSREEVVSELGNGIAAFESVPTAIYCFLRCMEPHPEIPSTFNSLQRTLIYSISLGGDTDTIATMAGAIAGAYYGMEQVPESWQQSCEGFEETDVLAQSLHRVFQESS